(19) United States
(12) Patent Application Publication
(10) Pub. No.: US 2010/0212037 A1
(43) Pub. Date: Aug. 19, 2010

(54) MIA-2 PROTEIN

(76) Inventors: Anja Katrin Bosserhoff, Regensburg (DE); Claus Hellerbrand, Regensburg (DE); de Buettner, Wachtberg-Pech (DE)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/634,281

(22) Filed: Dec. 9, 2009

Related U.S. Application Data
(63) Continuation-in-part of application No. 10/283,686, filed on Oct. 30, 2002, now abandoned.

(30) Foreign Application Priority Data
Oct. 16, 2002 (DE) 102 48 248.9

(51) Int. Cl. A61K 39/395; C07K 14/435; C07H 21/04; C12N 15/63; C12N 5/10; C12N 1/19; C12N 1/21; C07K 16/18; C07K 17/00; C12N 5/12; A61K 31/7088; A61K 38/17; A01K 67/027; A61P 35/00; C12Q 1/68; A61K 35/407; A61P 1/16

(52) U.S. Cl. 800/18; 530/350; 536/23.5; 536/24.1; 536/24.5; 536/24.3; 536/24.33; 435/320.1; 435/325; 435/419; 435/254.2; 435/348; 435/252.33; 435/252.31; 530/387.9; 530/387.3; 530/391.3; 530/391.7; 435/346; 514/44 R; 514/12; 424/139.1; 800/14; 435/6; 424/93.7

(57) ABSTRACT

The present invention relates to the human and murine melanoma inhibitory activity protein-2 (MIA-2) and to the nucleic acids encoding said proteins including a method for producing such proteins by recombinant techniques. The invention also relates to methods for utilizing such proteins for tissue regeneration, tumor treatment including to control the proliferation and differentiation of liver cells in vivo and in vitro. The invention further relates to diagnostic assays including the human and murine antibodies or aptamers and their use in therapy and diagnosis. Further it relates to diagnostic assays applying specific primers for the diagnostic of liver disease.

### CROSS REFERENCE TO RELATED APPLICATIONS

This application is a continuation-in-part of U.S. patent application Ser. No. 10/283,686, filed Oct. 30, 2002, which claims the benefit of German Patent Application No. 102 48 248.9 filed Oct. 16, 2002; the disclosures of each of which are incorporated herein by reference in their entireties.

### BACKGROUND OF THE INVENTION

1. Field of the Invention

The present invention relates to the isolation, production and use of MIA-2 protein and the nucleic acids encoding same especially for the use in liver disease, e.g. hepatitis, liver fibrosis or hepatocellular carcinoma. Compositions for such treatment comprise pharmaceutically acceptable compositions of MIA-2, alone or in combination. In accordance with another aspect the present invention relates to the use of MIA-2 sequences, antibodies or aptamers for the use in therapy and diagnostic of liver diseases like hepatitis, liver fibrosis or hepatocellular carcinoma. According to still another aspect the present invention relates to a process to develop organ cultures and their use in blood cleansing.

2. Description of Related Art

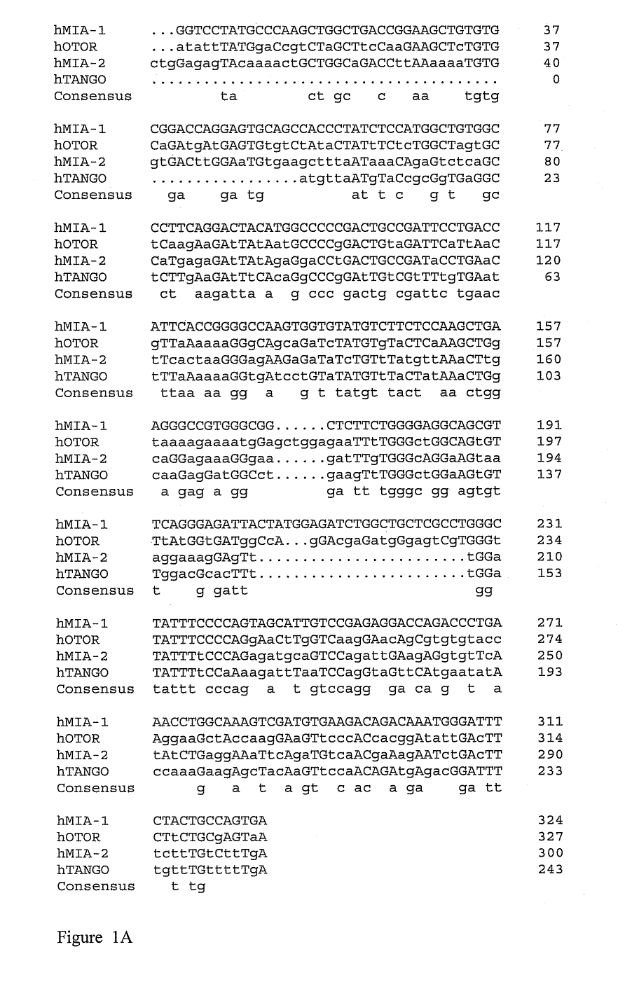

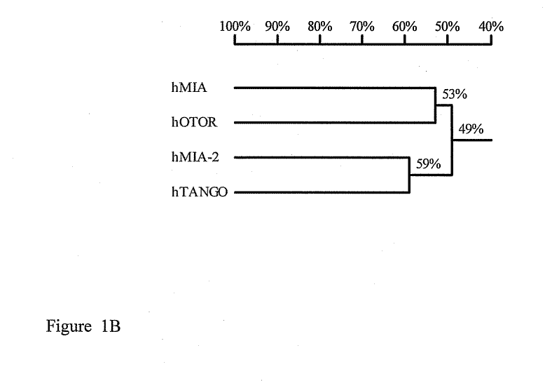

The protein MIA (“melanoma inhibitory activity”, also called CD-RAP “cartilage-derived retinoic acid-sensitive protein”) is expressed in chondrocytes and was originally isolated due to its anti-proliferative properties in vitro. Originally it was detected in cell culture supernatant of melanoma cells and isolated there from. After purification and partial sequencing of the protein, a human MIA cDNA fragment was isolated with the help of degenerated primers and RT-PCR (reverse transcriptase polymerase chain reaction). This fragment of 250 nucleic acid residues was used as a probe to screen a phage library to isolate the full length MIA cDNA clone (Blesch et al., 1995). A database search at that time using the full length MIA sequence did not reveal any homologous, known gene sequences. Now the sequences for humane, murine, bovine, rat and Zebra fish of MIA are known. The homology within the proteins is very high, indicating that MIA is highly conserved during evolution (FIG. 1).

The obtained cDNA sequence supported that MIA is translated as a 131 amino acid precursor protein. The signal sequence has a hydrophobic region containing 24 amino acids, which is important for the transport of the protein into the endoplasmatic reticulum (ER) and is cleaved off there. MIA is secreted into the extracellular space. The mature protein consists of 107 amino acids and has a molecular weight of about 11 kDa. Further analyses of the protein sequence showed that MIA has besides the signal sequence another four highly hydrophobic region stabilized by two intramolecular disulfide bridges, forming a globular structure. MIA does not contain amino acid series, (Asn-Gly-Ser/Thr; Ser-Gly), which are normally glycosylated, suggesting that there is no N- or O-glycosylation.

To elucidate the function of MIA during cartilage development and functional characterization, MIA-deficient mice were developed using the “knock-out” technology (Moser et al., 2002 Mol Cell Biol. 2002 March; 22(5):1438-45). MIA-deficient mice display changes in the cartilage organization and architecture. Further studies are ongoing to study the effect on integrity and stability of the cartilage.

Recently the MIA-homologous protein OTOR (MIAL, FPD) was characterized (Cohen-Salmon et al., 2000; Rendtorff et al., 2001; Robertson et al., 2000). OTOR is specifically expressed in the cochlea and eye. The inventor analyzed the expression of OTOR in MIA-deficient mice and could not detect a change in OTOR RNA levels. (Moser et al., 2002 Mol Cell Biol. 2002 March; 22(5):1438-45).

Subject-matter of EP 0 909 954 is a method for the diagnosis of cartilage diseases using the detection of MIA, a proper reagent, as well as the use of antibodies against MIA to detect cartilage diseases. MIA-2 sequences of their use have not been mentioned.

### SUMMARY OF THE INVENTION

It is an object of the present invention to provide a novel MIA protein, which can be advantageously used in the diagnosis and therapy of several kinds of liver damages.

This object is solved by the subject-matter of the independent claims. Preferred embodiments are set forth in the dependent claims.

Surprisingly, human and murine MIA-2 cDNA sequences have been identified using database searches and applying degenerated PCR to isolated MIA-2 cDNA fragments. Further characterization leads to the identification to first a truncated form of human and murine MIA-2, which was submitted to Genbank by the inventors (Bosserhoff, A. K. and Buettner, R; NCBI-Genbank, November 2001). These sequences contain 1-354 bp of the human MIA-2 sequence and 1-357 bp of the murine MIA-2 sequence. The sequences submitted to Genbank end with the stop codon TAA.

More surprisingly it has been discovered that the coding MIA-2 sequence and the corresponding protein is much longer compared to the sequence submitted to Genbank by the inventors. The stop codon at nucleotide position 352 for the human sequence and at nucleotide position 355 for the murine sequence is an artifact. Unexpectedly, the longer full length clones for MIA-2 were isolated and characterized. Very surprisingly, MIA-2 is far bigger compared to MIA.

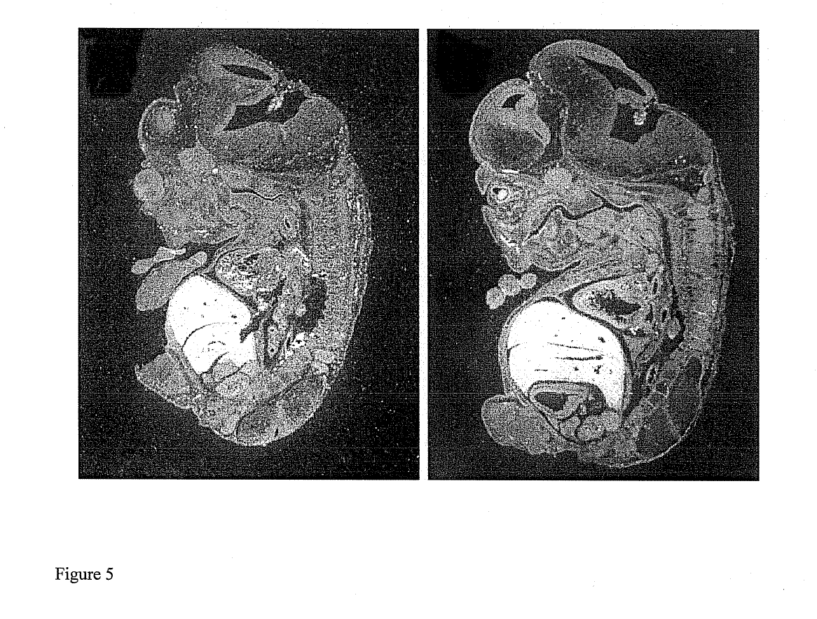

First experiments of the inventors showed that MIA-2 is selectively expressed in the liver of mouse embryos (FIG. 5). This liver-specific expression could be also confirmed for humans. More detailed analysis revealed that MIA-2 is mainly expressed in hepatocytes. The MIA-2 protein content in the liver serves as a measure for hepatic tissue damages, as well as a measure for synthesis performance. Experiments with a) tissue culture supernatants from cells transfected with a MIA-2 expression construct and b) with recombinant MIA-2 protein revealed that MIA-2 inhibits the proliferation of Ito cells and acts anti-fibrotic (the activation of Ito cells is the key event of the hepatic fibrosis and cirrhosis). Similar pilot studies with fibroblasts show as well the inhibition of proliferation. This points to a general mechanism not only limited to the liver and to potential use of MIA-2 in non-hepatic tissue. In in vitro assays it was possible to show that a number of cytokines, especially TGF-beta and interleukin 6, or physical stimulation induce MIA-2 expression at the RNA level in hepatic cells and Ito cells. These experiments demonstrate that MIA-2 expression could be increased either by a change of the cytokine environment or by physical stimuli possibly caused by overgrowth due to liver tumors or metastasis.

According to a first aspect, the present invention provides the human MIA-2 protein which is encoded by the nucleic acid of SEQ ID NO. 1 or variants thereof, which variants are defined as having one or more substitutions, insertions and/or deletions as compared to the nucleic acid of SEQ ID NO. 1 provided that
a) these variants hybridize under moderate stringent conditions to a nucleic acid which comprises the full or part of the sequence of SEQ ID NO. 1 and further provided that these variants code for a protein having MIA-2 activity; or
b) these variants have nucleic acid changes which can be deducted to the degeneration of the genetic code and code for the same or functional equivalent amino acid as the nucleic acid of SEQ ID NO. 1

According to a further aspect, the present invention provides the murine MIA-2 protein which is encoded by the nucleic acid of SEQ ID NO. 27 or variants thereof, which variants having one or more substitutions, insertions and/or deletions as compared to the nucleic acid of SEQ ID NO. 27 provided that

a) these variants hybridize under moderate stringent conditions to a nucleic acid which comprises the full or part of the sequence of SEQ ID NO. 27 and further provided that these variants code for a protein having MIA-2 activity
b) these variants have nucleic acid changes which can be deducted to the degeneration of the genetic code and code for the same or functional equivalent amino acid as the nucleic acid of SEQ ID NO. 27.

The invention further provides a human, isolated nucleic acid, which comprises the nucleic acid of SEQ ID NO. 1 or variants thereof, wherein the variants are each defined as having one or more substitutions, insertions, and/or deletions as compared to the nucleic acid of SEQ ID NO. 1, provided that:
a) these variants hybridize under moderate stringent conditions to a nucleic acid, which comprises the sequence of SEQ ID NO. 1, and further provided that these variants code for a protein having MIA-2 activity; or
b) said variants having nucleic acid changes which are due to the degeneration of the genetic code, which code for the same or functional equivalent amino acids as the nucleic acid of SEQ ID NO. 1.

Further, the invention provides an isolated nucleic acid which comprises the nucleic acid of SEQ ID NO. 27 or variants thereof, wherein the variants are each defined as having one or more substitutions, insertions, and/or deletions as compared to the sequence of SEQ ID NO. 27, provided that:
a) said variants hybridize under moderate stringent conditions to a nucleic acid, which comprises the sequence of SEQ ID NO. 27, and further provided that these variants code for a protein having MIA-2 activity; or
b) these variants having nucleic acid changes, which are due to the degeneration of the genetic code, which code for the same or a functional equivalent amino acid as compared to the nucleic acid of SEQ ID NO. 27.

The nucleic acid variants according to the invention comprise nucleic acid fragments which contain more than 10, preferably more than 15, more than 20, more than 25 or more than 30 and up to 50 nucleotides. The term oligonucleotide includes fragments containing 10 to 50 nucleotides and parts thereof. These sequences can be in any order as long as at least 10 successive nucleotides are according to the invention. These oligonucleotides can be preferably used as primer, for example for RT-PCR or as a probe for in situ hybridization. According to a preferred embodiment, a fragment of the MIA-2 nucleic acids of the present invention is defined as bases 1-354 of SEQ ID NO: 1 for the human MIA-2 sequence and bases 1-357 of SEQ ID NO: 27 for the murine MIA-2 sequence. In other words, said nucleic acid sequences code for a human MIA-2 protein comprising the amino acids 1-118 of SEQ ID NO: 5 and a murine MIA-2 protein comprising amino acids 1-119 of SEQ ID NO: 28, respectively.

In some embodiments, the present invention provides MIA-2 protein variants which do not comprise amino acids 1 to 19 of SEQ ID NO:1. In some embodiments the N-terminal amino acids 1 to 19 of human MIA-2 protein form a signal peptide. In one embodiment of the invention these protein variants start at the N-terminus with the amino acids LEST (1-letter code). In another embodiment these proteins have an additional methionine at the N-terminus such that the N-terminal sequence is MLEST.

In some embodiments of the invention, the MIA-2 protein variants are defined by the amino acid sequence of SEQ ID NO:29 or SEQ ID NO:30. In some embodiments the sequences of SEQ ID NO:29 or SEQ ID NO:30 comprise an additional serine at position 83, which is not part of the human MIA-2 sequence disclosed in the subject application. In some embodiments the MIA-2 variants described herein comprise this additional serine, and the nucleic acids encoding these variants comprise the corresponding additional serine codon.

In a further embodiment, the present invention includes protein variants which comprise the amino acid sequence of SEQ ID NO:29 or SEQ ID NO:30 and up to 10, and in some embodiments up to 5, additional amino acids at the N- or C-terminus, or variant of these amino acid sequences, wherein said variants contain one or more substitutions, insertions and/or deletions when compared to the amino acid sequence of SEQ ID NO:29 or SEQ ID NO:30, and wherein the biological activity is at least substantially equal to the activity of the MIA-2 protein. Preferably, the sequence identity of such variants is at least about 70, 80, 90 or 95% identical to the sequence of SEQ ID NO:29 or SEQ ID NO:30.

In some embodiments the present invention provides nucleic acids encoding any of these variants or fragments. In some embodiments the present invention provides an isolated nucleic acid which comprises the nucleic acid of SEQ ID NO. 31 or SEQ ID NO:32, or variants thereof, wherein the variants are each defined as having one or more substitutions, insertions, and/or deletions as compared to the sequence of SEQ ID NO. 31 or SEQ ID NO:32, provided that:
a) said variants hybridize under moderately stringent conditions to a nucleic acid, which comprises the sequence of SEQ ID NO:31 or 32, and further provided that these variants code for a protein having MIA-2 activity; or
b) these variants have nucleic acid changes, which are due to the degeneration of the genetic code, which code for the same or a functional equivalent amino acid as compared to the nucleic acid of SEQ ID NO:31 or 32.

In general, the functions, applications and variants of the protein fragment and nucleic acid fragments which lack the N-terminal signal sequence are characterized as outlined above for the full length MIA-2 protein and nucleic acids. Thus what is said above relating to the MIA-2 protein and nucleic acid also relates to these fragments.

The protein variants without the signal sequence are especially useful for the therapy and prevention of liver diseases, such as liver fibrosis.

According to a preferred embodiment, a fragment of the MIA-2 nucleic acids of the present invention is defined as bases 58-357 of SEQ ID NO:1 (Fragment SPR30-03) or bases 58-759 of SEQ ID NO:1 (Fragment SPR30-04) of the human MIA-2 sequence. In other words, said nucleic acid sequences code for a human MIA-2 protein comprising the amino acids 20-119 of SEQ ID NO:5 (Fragment SPR30-03) and amino acids 20-253 of SEQ ID NO:5 (Fragment SPR30-04).

Subject of the invention is also a pharmaceutical composition which contains MIA-2 protein or fragments of the MIA-2 protein such as SPR30-03 or SPR30-04.

According to the state of the art an expert can test which derivatives and possible variations derived from these revealed nucleic acid sequences according to the invention are, are partially or are not appropriate for specific applications like hybridization and PCR assays. The nucleic acid and oligonucleotides of the inventions can also be part of longer DNA or RNA sequences, e.g. flanked by restriction enzyme sites.

Amplification and detection methods are according to the state of the art. The methods are described in detail in protocol books which are known to the expert. Such books are for example Sambrook et al., 1989, Molecular Cloning, A Laboratory Manual, Cold Spring Harbor Press, and all subsequent editions. PCR-methods are described for example in Newton, PCR, BIOS Scientific Publishers Limited, 1994 and all subsequent editions.

As defined above, “variants” are according to the invention especially such nucleic acids, which contain one or more substitutions, insertions and or deletions when compared to the nucleic acids of SEQ ID No. 1 and 27. These lack preferably one, but also 2, 3, 4, or more nucleotides 5′ or 3′ or within the nucleic acid sequence, or these nucleotides are replaced by others.

The nucleic acid sequences of the present invention comprise also such nucleic acids which contain sequences in essence equivalent to the nucleic acids described in SEQ ID No. 1 and 27. According to the invention nucleic acids can show for example at least about 80%, more typically at least about 90% or 95% sequence identity to the nucleic acids described in SEQ ID No. 1 and 27.

The term “nucleic acid sequence” means a heteropolymer of nucleotides or the sequence of these nucleotides. The term “nucleic acid”, as herein used, comprises RNA as well as DNA including cDNA, genomic DNA and synthetic (e.g. chemically synthesized) and to other polymers linked bases such as PNA (peptide nucleic acids).

The invention comprises—as mentioned above—also such variants which hybridize to the nucleic acids according to the invention at moderate stringent conditions.

Stringent hybridization and wash conditions are in general the reaction conditions for the formation of duplexes between oligonucleotides and the desired target molecules (perfect hybrids) or that only the desired target can be detected. Stringent washing conditions mean 0.2×SSC (0.03 M NaCl, 0.003 M sodium citrate, pH 7)/0.1% SDS at 65° C. For shorter fragments, e.g. oligonucleotides up to 30 nucleotides, the hybridization temperature is below 65° C., for example at 50° C., preferably above 55° C., but below 65° C. Stringent hybridization temperatures are dependent on the size or length, respectively of the nucleic acid and their nucleic acid composition and will be experimentally determined by the skilled artisan. Moderate stringent hybridization temperatures are for example 42° C. and washing conditions with 0.2×SSC/0.1% SDS at 42° C.

The respective temperature conditions can vary dependent on the chosen experimental conditions and to be tested nucleic acid probe, and have to be adapted appropriately. The detection of the hybridization product can be done for example using X-Ray in the case of radioactive labeled probes or by fluorimetry in the case of fluorescent labeled probes.

The expert can according to the state of the art adapt the chosen procedure, to reach actually moderate stringent conditions and to enable a specific detection method. Appropriate stringent conditions can be determined for example on the basis of reference hybridization. An appropriate nucleic acid or oligonucleotide concentration needs to be used. The hybridization has to occur at an appropriate temperature (the higher the temperature the lower the binding).

Fragments of the nucleic acids according to the invention can be used for example as oligonucleotide primer in detection systems and amplification methods of the MIA-2 gene and MIA-2 transcript. The expert can apply these oligonucleotides in state of the art methods. DNA or RNA can be analyzed for the presence of one of the described genes or transcripts applying the appropriate oligonucleotide primers to the to be analyzed probe. The detection of the RNA or DNA of the probe can be achieved for example by PCR methods, which reveal the presence of the specific DNA and/or RNA sequences. All hereinabove described oligonucleotides can also be used as primers, also as primers for reverse transcription of RNA.

The PCR method has the advantage that very small amounts of DNA are detectable. Dependent on the to be analyzed material and the equipment used the temperature conditions and number of cycles of the PCR have to be adjusted. The optimal conditions can be experimentally determined according to standard procedures.

The during the PCR amplification accrued, characteristic, specific DNA fragments can be detected for example by gel electrophoretic or fluorimetric methods with the DNA labeled accordingly. Alternatively, other appropriate, known to the expert, detection systems can be applied.

The DNA or RNA, especially mRNA, of the to be analyzed probe can be an extract or a complex mixture, in which the DNA or RNA to be analyzed are only a very small fraction of the total biological probe. This probe can be analyzed by PCR, e.g. RT-PCR or in hybridization assays. The biological probe can be serum, blood or cells, either isolated or for example as mixture in a tissue. Further, the herein described oligonucleotides can be used for RT-PCR, in situ PCR or in situ hybridization.

In the case of RT-PCR oligonucleotides of the invention are used for PCR amplification of fragments of cDNA matrices, which resulted from the reverse transcription of probe RNA or mRNA. The expression analysis can be qualitative or together with appropriate controls and methods quantitative. For the quantitative analyses an internal standard is used.

According to an embodiment of the invention, the isolated nucleic acid according to the invention is further operably linked to one or more regulatory sequences. Especially, the human MIA-2 promoter according to SEQ ID NO. 2 is preferred here. A specially preferred region of the promoter, which still functions specifically in the liver, contains the base pairs 2241-3090 of SEQ ID NO. 2.

The present invention comprises further transcriptional products of the hereinabove described nucleic acids and nucleic acids, which selectively hybridize under moderate stringent conditions to one of these transcriptional products. Preferably this comprises an antisense DNA or RNA in form of a DNA or RNA probe which can hybridize to a transcription product, e.g. mRNA, and can be used in detection systems.

The term “probe” is here defined as a nucleic acid which can bind to a target nucleic acid via one or more kind of chemical binding, usually via complementary base pairing which usually utilizes hydrogen bonds.

For detection the nucleic acids according to the invention are preferably labeled, for example with radioactive labellings, digoxygenin, biotin, peroxidase, fluorescence or alkaline phosphatase. Depending on the label, the detection can be direct or enhanced using indirect immunohistochemistry. Alkaline phosphatase is used as marker enzyme since it develops a sensitive, striking color reaction in the presence of appropriate substrates. Substrates, like p-nitrophenylphosphate, are cleaved and release colored, photometrically measurable products.

In a further embodiment, the present invention provides nucleic acids coupled to a matrix, e.g. nylon membrane, glass or polymers.

For the amplification of the human nucleic acid according to SEQ ID NO. 1 and variants thereof or transcriptional products thereof, one can apply the forward and reverse primers according to SEQ ID NO. 3, 4, 9 or 26 besides the hereinabove described primer. Analogous for the amplification of the murine nucleic acid according to SEQ ID NO. 27 and variants thereof or transcriptions products thereof, one can apply the forward and reverse primers according to SEQ ID NO. 3, 7, 9 or 26. For the amplification of the MIA-2 promoter one or more nucleic acids can be applied according to SEQ ID NO. 10-18.

In a further embodiment, the present invention includes human MIA-2 protein which comprises the amino acid according to SEQ ID NO. 5 or a variant of this amino acid, wherein said variants contain one or more substitutions, insertions and/or deletions when compared to the amino acid sequence of SEQ ID NO. 5, and wherein the biological activity is substantially equal to the activity of the MIA-2 protein. Variants of the protein can also be N-terminal or C-terminal truncations of SEQ ID NO. 5, especially the variant containing amino acid residues 1-119.

In particular variants of the protein, for example deletions, insertions and/or substitutions in the sequence, which cause for so-called “silent” changes, are considered to be part of the invention.

For example, such changes in the nucleic acid sequence are considered to cause a substitution with an equivalent amino acid. Preferably are such amino acid substitutions the result of substitutions which substitute one amino acid with a similar amino acid with similar structural and/or chemical properties, i.e. conservative amino acid substitutions. Amino acid substitutions can be performed on the basis of similarity in polarity, charges, solubility, hydrophobic, hydrophilic, and/or amphipathic (amphiphile) nature of the involved residues. Examples for hydrophobic amino acids are alanine, leucine, isoleucine, valine, proline, phenylalanine, tryptophan and methionine. Polar, neutral amino acids include glycine, serine, threonine, cysteine, thyronine, asparagine and glutamine. Positively (basic) charged amino acids include arginine, lysine and histidine. And negatively charged amino acids include aspartic acid and glutamic acid.

“Insertions” or “deletions” usually range from one to five amino acids. The allowed degree of variation can be experimentally determined via methodically applied insertions, deletions or substitutions of amino acids in a polypeptide molecule using recombinant DNA methods. The resulting variants can be tested for their biological activity.

Nucleotide changes, which affect the N-terminal and C-terminal part of the protein, often do not change the protein activity, because these parts are often not involved in the biological activity. It can be desired to eliminate one or more of the cysteins of the sequence, since cysteines can cause the unwanted formation of multimers when the protein is produced recombinant. Multimers may complicate purification procedures. Each of the suggested modifications is in range of the current state of the art, and under the retention of the biological activity of the encoded products.

In a further embodiment, the present invention includes the invention of a vector (construct) comprising a nucleic acid according to the invention. This vector is preferably an expression vector which contains a nucleic acid according to the invention and one or more regulatory nucleic acid sequences.

Numerous vectors are known to be appropriate for the transformation of bacterial cells, for example plasmids and bacteriophages, like the phage λ, are frequently used as vectors for bacterial hosts. Viral vectors can be used in mammalian and insect cells to express exogenous DNA fragments, e.g. SV 40 and polyoma virus.

The transformation of the host cell can be done alternatively directly using “naked DNA” without the use of a vector.

The protein according to the invention can be produced either in eukaryotic or prokaryotic cells. Examples for eukaryotic cells include mammalian, plant, insect and yeast cells. Appropriate prokaryotic cells include Escherichia coli and Bacillus subtilis.

Preferred mammalian host cells are CHO, COS, HeLa, 293T, HEH or BHK cells or adult or embryonic stem cells.

Alternatively, the protein according to the invention can be produced in transgenic plants (e.g. potatoes, tobacco) or in transgenic animals, for example in transgenic goats or sheep.

In a further embodiment, the present invention includes an antibody or aptamer which recognizes MIA-2 protein according to the invention.

The antibody is preferably selected from a group, which consists of polyclonal antibodies, monoclonal antibodies, humanized antibodies, chimeric antibodies and synthetic antibodies.

The antibody according to the invention can be additionally linked to a toxic and/or a detectable agent.

The term “antibody”, is used herein for intact antibodies as well as antibody fragments, which have a certain ability to selectively bind to an epitop. Such fragments include, without limitations, Fab, F(ab′)₂ and Fv antibody fragment. The term “epitop” means any antigen determinant of an antigen, to which the paratop of an antibody can bind. Epitop determinants usually consist of chemically active surface groups of molecules (e.g. amino acid or sugar residues) and usually display a three-dimensional structure as well as specific physical properties.

The antibodies according to the invention can be produced according to any known procedure. For example the pure complete protein according to the invention or a part of it can be produced and used as immunogen, to immunize an animal and to produce specific antibodies.

The production of polyclonal antibodies is commonly known. Detailed protocols can be found for example in Green et al, Production of Polyclonal Antisera, in Immunochemical Protocols (Manson, editor), pages 1-5 (Humana Press 1992) and Coligan et al, Production of Polyclonal Antisera in Rabbits, Rats, Mice and Hamsters, in Current Protocols In Immunology, section 2.4.1 (1992). In addition, the expert is familiar with several techniques regarding the purification and concentration of polyclonal antibodies, as well as of monoclonal antibodies (Coligan et al, Unit 9, Current Protocols in Immunology, Wiley Interscience, 1994).

The production of monoclonal antibodies is as well commonly known. Examples include the hybridoma method (Kohler and Milstein, 1975, Nature, 256:495-497, Coligan et al., section 2.5.1-2.6.7; and Harlow et al., Antibodies: A Laboratory Manual, page 726 (Cold Spring Harbor Pub. 1988).), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

In brief, monoclonal antibodies can be attained by injecting a mixture which contains the protein according to the invention into mice. The mice used can be also a transgenic mouse or a mouse deficient in MIA-2. The antibody production in the mice is checked via a serum probe. In the case of a sufficient antibody titer, the mouse is sacrificed and the spleen is removed to isolate B-cells. The B cells are fused with myeloma cells resulting in hybridomas. The hybridomas are cloned and the clones are analyzed. Positive clones which contain a monoclonal antibody against the protein are selected and the antibodies are isolated from the hybridoma cultures. There are many well established techniques to isolate and purify monoclonal antibodies. Such techniques include affinity chromatography with protein A sepharose, size-exclusion chromatography and ion exchange chromatography. Also see for example, Coligan et al., section 2.7.1-2.7.12 and section “Immunglobulin G (IgG)”, in Methods In Molecular Biology, volume 10, pages 79-104 (Humana Press 1992).

According to a still further embodiment, the invention as hereinabove described provides a hybridoma cell line which produces a monoclonal antibody which specifically binds to MIA-2 protein according to the invention.

The invention further includes a pharmaceutical composition comprising a nucleic acid according to the invention, a vector, protein, antibody or aptamer according to the invention as an active component in combination with a pharmaceutical acceptable carrier.

The active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutical acceptable” is defined as non-toxic material, which does not interfere with effectiveness of the biological activity of the active component. The choice of the carrier is dependent on the application.

The pharmaceutical composition can contain additional components which enhance the activity of the active component or which supplement the treatment. Such additional components and/or factors can be part of the pharmaceutical composition to achieve a synergistic effects or to minimize adverse or unwanted effects.

Techniques for the formulation or preparation and application/medication of compounds of the present invention are published in “Remington's Pharmaceutical Sciences”, Mack Publishing Co., Easton, Pa., latest edition. A therapeutically effective dose relates to the amount of a compound which is sufficient to improve the symptoms, for example a treatment, healing, prevention or improvement of such conditions. An appropriate application can include for example oral, dermal, rectal, transmucosal or intestinal application and parenteral application, including intramuscular, subcutaneous, intramedular injections as well as intrathecal, direct intraventricular, intravenous, intraperitoneal or intranasal injections. The intravenous injection is the preferred treatment of a patient.

A typical composition for an intravenous infusion can be produced such that it contains 250 ml sterile Ringer solution and for example 10 mg MIA-2 protein. See also Remington's Pharmaceutical Science (15. edition, Mack Publishing Company, Easton, Pa., 1980).

The active component or mixture of it in the present case can be used for prophylactic and/or therapeutic treatments.

In the case of therapeutic application a patient with liver damage, e.g. cirrhoses, fibrosis, hepatitis or hepatocellular carcinoma/metastasis, will be treated. The nucleic acids/proteins according to the invention are appropriate to treat liver damage, like liver cirrhoses and fibrosis. The MIA-2 gene according to the invention and the corresponding amino acid sequence of the MIA-2 protein of the present invention inhibit proliferation especially of liver cells, but possibly also in other tissues like spleen or intestine (see table 1). For more detailed information see the examples.

An amount which is adequate to reach the aforesaid effect is defined as “therapeutically effective dose”. Amounts, which are effective for these applications, depend on the severity of the condition and the general condition of the patient and his immune system. However, the dose range is usually between 0.01 and 100 mg protein per dose with a dose of 0.1 to 50 mg and from 1 to 10 mg per patient. Single or multiple applications after a daily, weekly or monthly treatment regimen can be performed with application rate and samples chosen by the physician in charge.

A pharmaceutical composition which contains MIA-2 protein according to the invention in combination with a pharmaceutical compatible carrier can either contain additional active compounds like interferons, inhibitors of the ACE-pathway or ligands of the proliferation-activated receptor-gamma (PPAR-g), which further support the anti-fibrotic effect of the MIA-2 protein.

In a further embodiment, the present invention includes a diagnostic composition which contains an antibody, aptamer or probe according to the invention.

Further, the invention includes a transgenic, non-human mammal, which has one or more MIA-2 sequences according to the inventions inactivated. Using the homologous recombination technology as described for example in “(Gene Targeting: A Practical Approach” (editor A. Joyner, Oxford University Press, 2nd edition, 2002) or “Gene Knockout Protocols” (editor M. J. Tymms and I. Kola, Humana Press, 1st edition 2001), a knock-out animal model can be established. This will enable to elucidate further functions of MIA-2 and especially the etiology of liver damage etc. Further, the knock-out animal may be suitable for the production of monoclonal antibodies.

The invention comprises preferably a transgenic mouse with a nucleic acid of the invention conditionally inactivated. This is a special case within the knock-out technology. The original knock-out technology applications result in the constitutively deletion of the gene to be analyzed. In the present invention a system will be used to create a cell type-specific and/or temporally controlled conditionally inactivation of a gene in a specific tissue or cell type at a specific time point. For the conditional gene inactivation in a certain tissue a specific promoter is necessary to disable the desired gene in the selected tissue or cells. For example the MIA-2 promoter according to the invention can be used to inactivate selected genes in the liver. To achieve this, the MIA-2 promoter according to the invention will be ligated at the DNA level to an appropriate recombinase, for example Cre of flp. This construct may further include other regulatory sequences to guarantee the expression of the recombinase. The construct can be tested in vitro before it is used to produce transgenic, non-human animals, preferably transgenic mice. The founder mice will be analyzed for correct expression of the recombinase in the specific tissue or cells, for example in liver, and the positive ones will be later used for intercrossing. Genes to be cell- or tissue-specific inactivated are cloned into vector such that the regions to be deleted are flanked by recombinase recognition sites, for example loxP for the Cre recombinase and frt for the Flp recombinase. Using the knock-out technology the vector is transfected into embryonic stem (ES) cells and clones with the correct integrations are selected and used for the production of chimeric animals. The heterozygous or homozygous offspring of these will be intercrossed with transgenic mice containing the recombinase resulting in a tissue-specific deletion of the selected gene. The effects can be analyzed and will lead to a further understanding of the liver metabolism. With the use of the MIA-2 promoter the effect of genes specifically in the liver can be analyzed leading to a greater understanding of liver homeostasis.

Further, the present invention provides a non-human transgenic mammal, which has a nucleic acid according to invention inserted. For example the MIA-2 cDNA can be ectopically expressed to investigate activities of MIA-2 in other tissues. Further the MIA-2 promoter nucleic acid according to the invention can be ligated to other cDNAs or genes and other regulatory sequences to overexpress these cDNAs or genes specifically in the liver. These will allow to study the function of these in the liver. This method can be applied for target identification and validation to develop potential novel treatments for liver diseases.

According to a further embodiment, the present invention comprises an ex vivo method to diagnose a liver damage or to determine the hepatic synthesis performance which includes the following steps
a) provide a liver biopsy or serum sample of a patient
b) qualitative and/or quantitative determination of the transcriptional products (especially mRNA) according to the invention of the MIA-2 gene in the sample, whereas a change in the transcription level indicates liver damage and/or increased hepatic activity.

The analysis in step b) is preferably done by Northern Blot, in situ hybridization or RT-PCR or a combination thereof. For further details see also McPherson et al. (ed.), PCR, A Practical Approach, Oxford, IRL Press 1995.

For RT-PCR the preferred primers are SEQ ID NO. 3 and 9 (human MIA-2) and SEQ ID NO. 3 and 7 (murine MIA-2).

Further the analysis in step b) can be done using a diagnostic composition as hereinabove described with anti MIA-2 antibodies or aptamers or using specific DNA or RNA probes for MIA-2 according to the invention.

Especially, the diagnostic method of the invention can be used for a potential liver damage like liver cirrhosis, fibrosis or hepatocellular carcinoma and metastasis.

The pharmaceutical compositions according to the invention are especially applied for the anti-fibrotic therapy as mentioned above, however, especially of the treatment of cirrhosis, fibrosis and/or hepatocellular carcinoma and metastasis.

According to a further embodiment, the present invention comprises a procedure for the manufacture of an organ culture, which includes the following steps:

a) supply mammalian hepatocytes in a media
b) add MIA-2 protein according to claim 1, 2, 16 or 17 to the mammalian hepatocytes
c) isolate the developed organ culture

In step a) human or porcine hepatocytes are preferably used.

The developed organ culture can be of advantage for the ex vivo blood cleansing for patients which do not have sufficient liver function due to liver damage.

The present invention will be further described with reference to the following figures and examples; however, it is to be understood that the present invention is not limited to such figures and examples.

FIG. 1 shows the comparison of human MIA, OTOR, MIA-2 and TANGO cDNA-sequences.
(a) Sequence alignment of the four human homologous MIA cDNA sequences.
(b) Homology of members of the MIA gene family at cDNA level, displayed as family tree. The tree was synthesized with the help of the software program DNAman and uses the coding region as basis for the alignment. The length of each horizontal line is proportional to the degree of the cDNA sequence divergence.

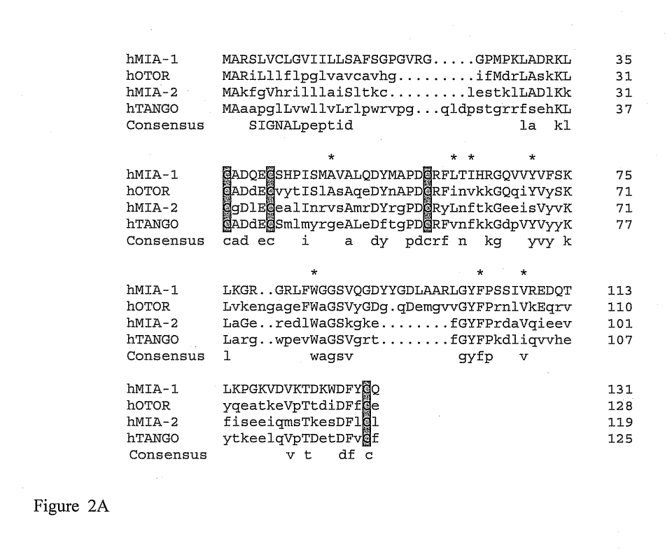

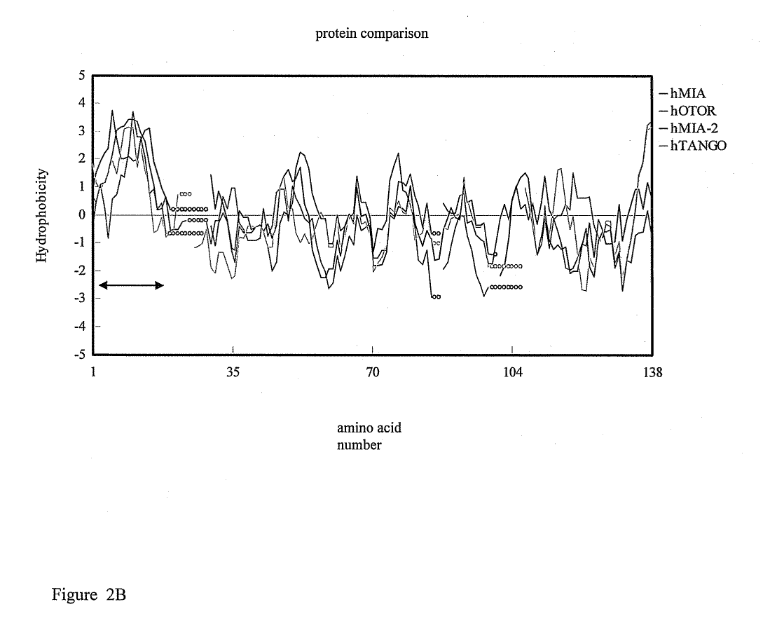

FIG. 2 shows a comparison of human MIA, OTOR, MIA-2 and TANGO peptide sequences
(a) Sequence alignment of the four human homologous MIA amino acid sequences. Conserved cysteine residues are marked with a box. The residues labeled with a star (*) are important for the hydrophobe nucleus of the SH3 domain.
(b) Kyte-Dolittle-Blot, which analyzes the hydrophobic characteristics of the homologous MIA proteins. The arrows indicate highly hydrophobic signal sequences.

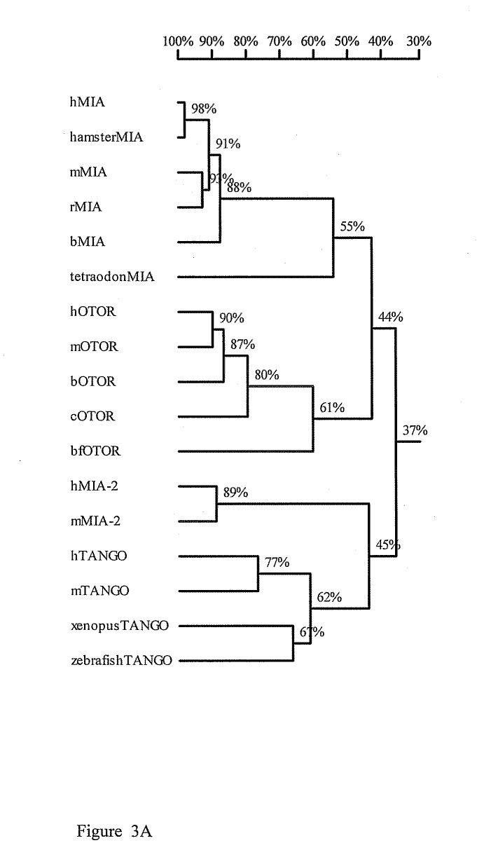

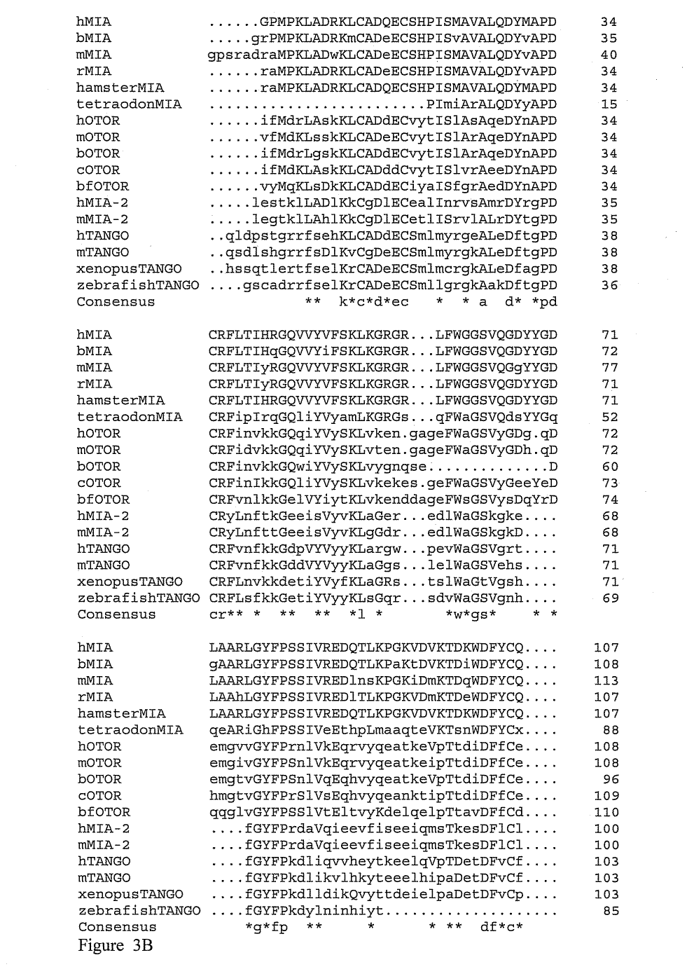

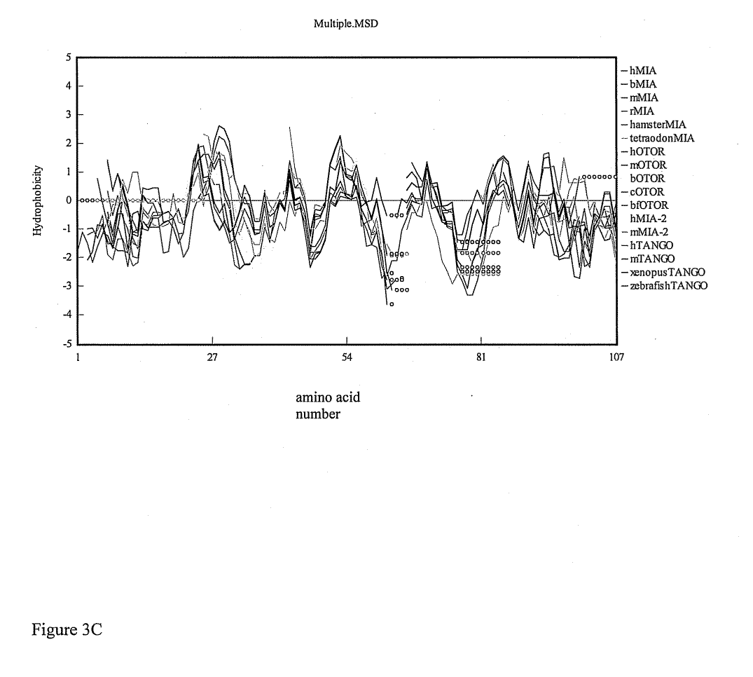

FIG. 3 shows a comparison of all available sequences of the MIA gene family
(a) The homology between all MIA gene family members at the protein level is displayed as family tree. The species are abbreviated such: h=humane, b=bovine, m=murine, r=rat, bf=Xenopus, c=chick. The tree was synthesized with the help of the software program DNAman and uses the deduced amino acid sequences as basis for the alignment. Since the N-terminal signal peptides vary highly, only the mature proteins were compared. For Tetraodin-MIA and Zebra fish-TANGO only partial sequences are available.
(b) Amino acid comparison of all available MIA gene family members. The amino acid sequences are displayed as single-letter-code, the numbers indicate the residues in relation to the initial amino acid residue of the mature protein without signal sequence. Identical residues are shown in the last lane, similarities are indicated by a star (*).
(c) A Kyte-Dolittle-Blot shows the highly conserved overall structure of the different species.

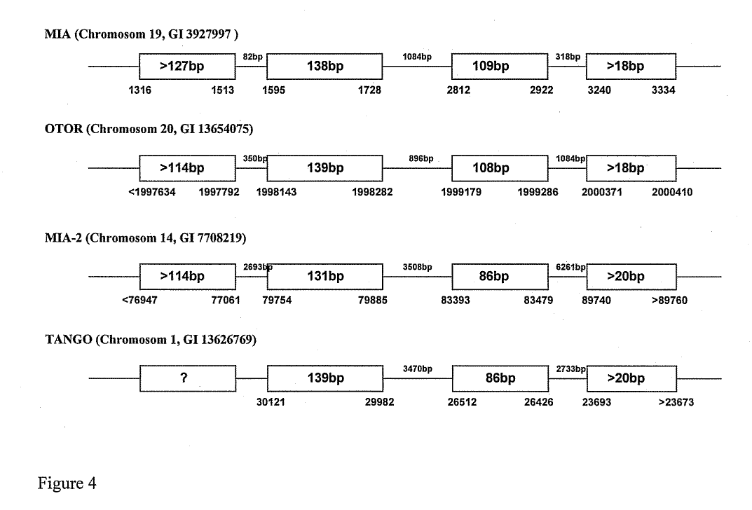

FIG. 4 shows the genomic organization of the human MIA gene family

The exon-intron structure was constructed by adapting the cDNA sequence to the equivalent genomic region. Exons and introns are indicated with boxes and lines. The number of the boxes shows the length of the exon. The humane genomic TANGO-sequence is incomplete.

FIG. 5 shows a RNA in situ-hybridization on sections of mouse embryos (embryonic stage day 12.5 and day 14.5).

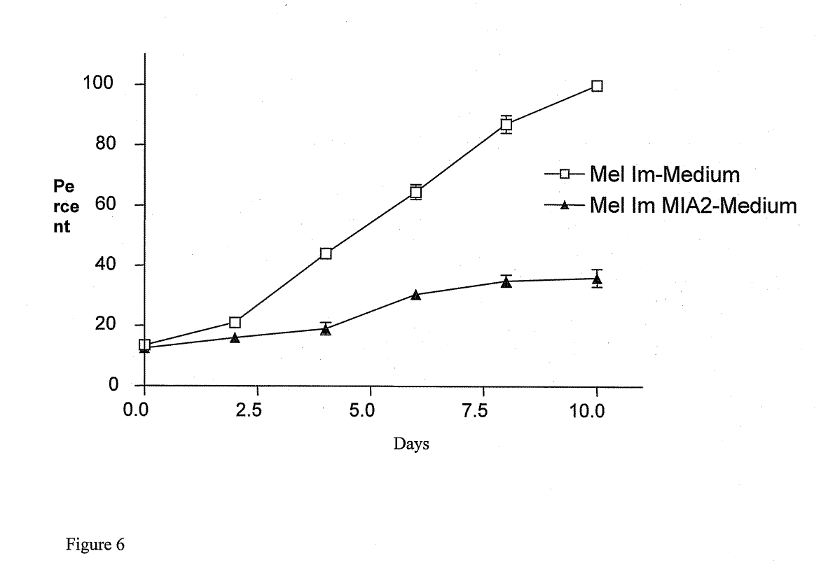

FIG. 6 shows the influence of MIA-2 on the proliferation of activated Ito cells.

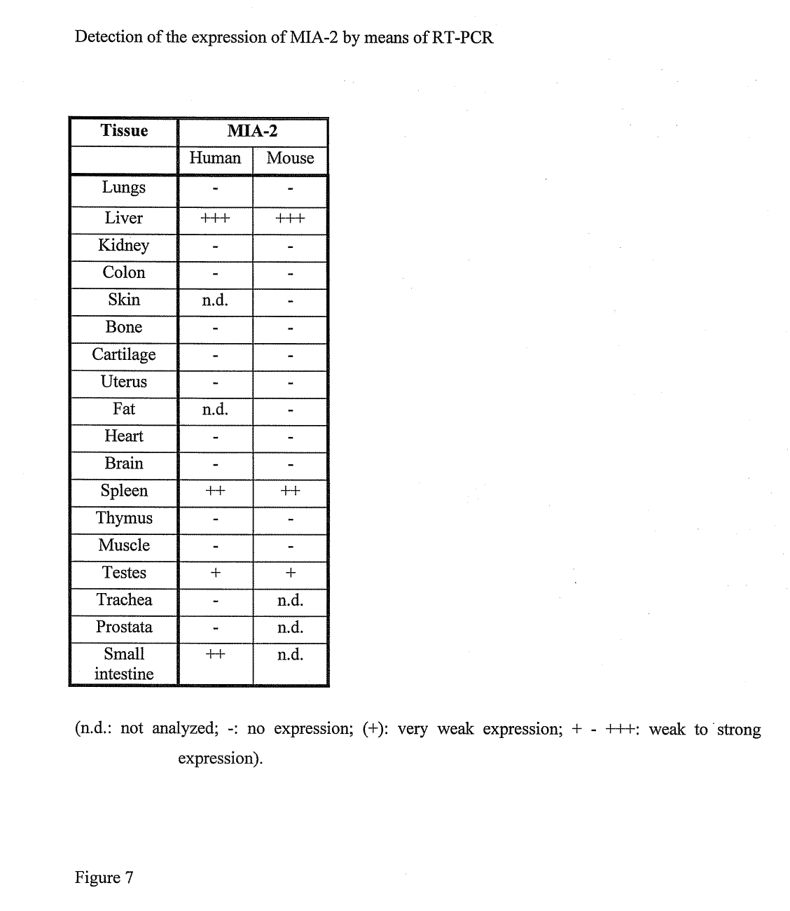

FIG. 7 shows the RNA expression of MIA-2 in different humane and murine tissues. The tissues were analyzed by RT-PCR.

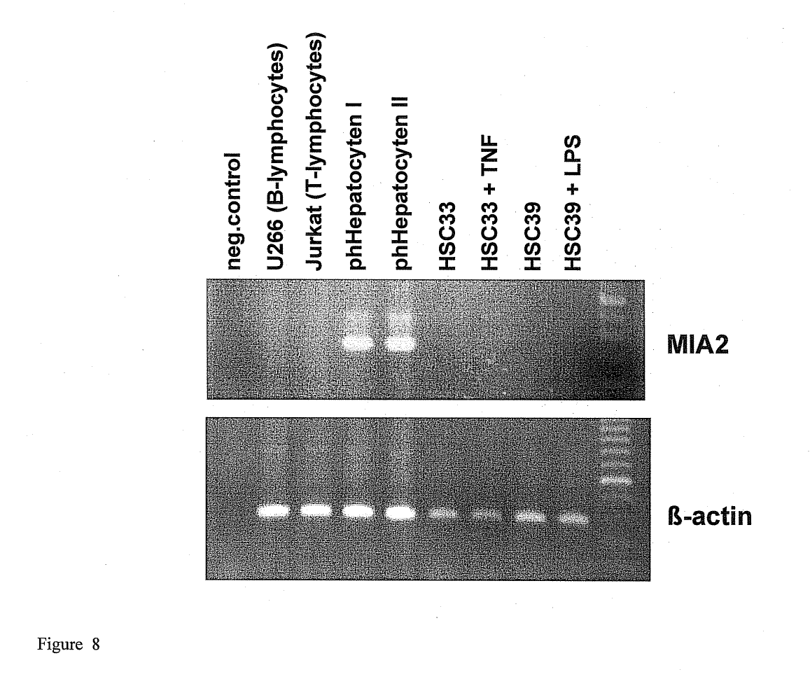

FIG. 8 shows the RNA expression of MIA 2 in primary human hepatocytes.

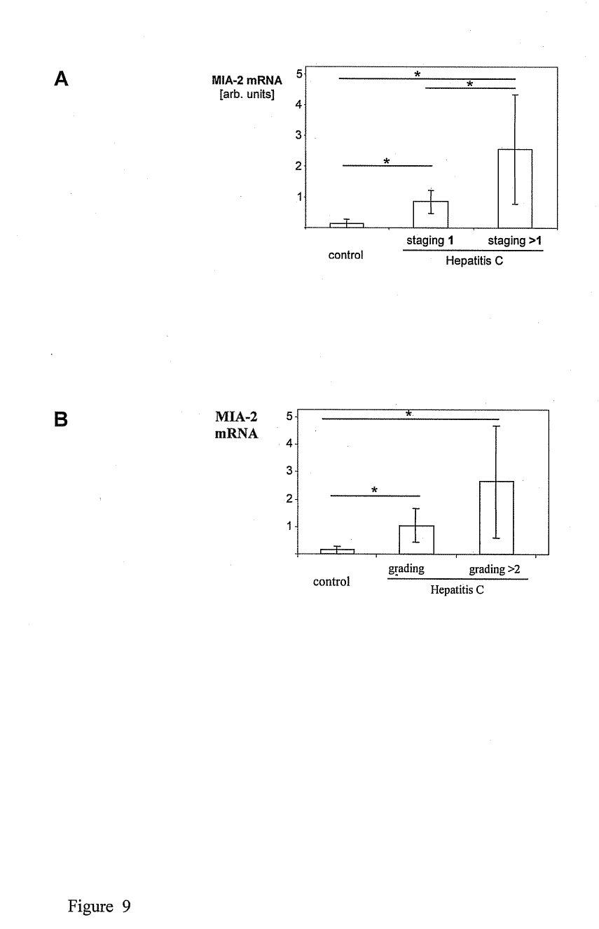

FIG. 9 shows that in biopsies from hepatitis patients with mild fibrosis MIA-2 RNA levels are significantly lower compared to biopsies from Hepatitis patients with progressed fibrosis.

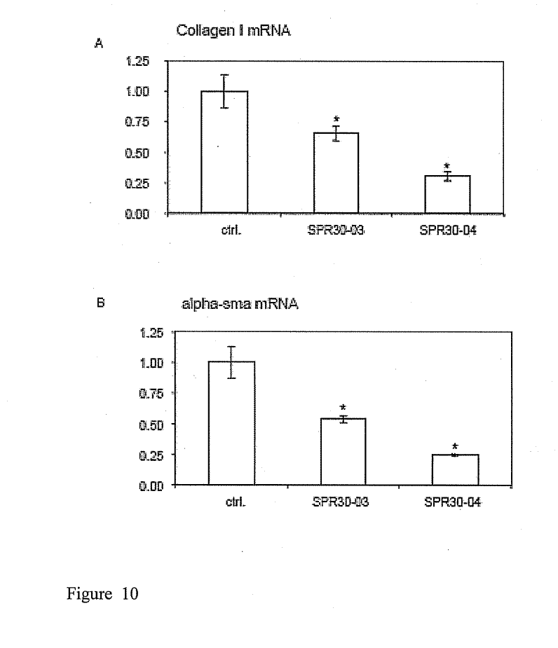

FIG. 10 shows the effect of two Mia-2 variants SPR30-03 and SPR30-04 on liver fibrosis, demonstrated by using an in vitro model for hepatic fibrosis. Shown is the expression of two different markers for the activation of hepatic stellate cells as a model for hepatic fibrosis. FIG. 10A shows the mRNA expression level of Collagen Type I (alpha 1) mRNA. FIG. 10B shows the mRNA expression level of alpha-smooth muscle actin (alpha-sma) mRNA.

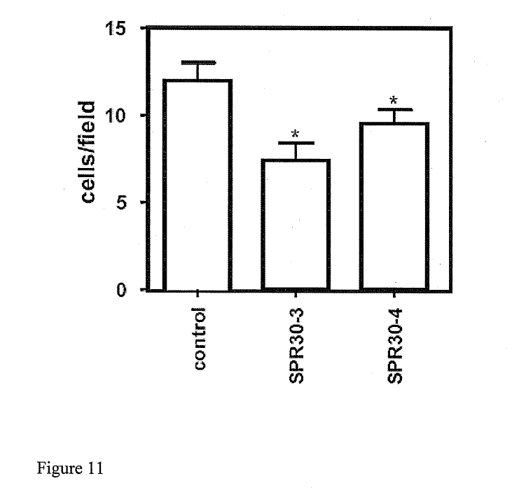

FIG. 11 shows the results of migration assay of HCC-cell line treated with rMIA2 (200 ng/ml for 4 h) applying Boyden chamber assays. Bars represent the number of cells counted on representative areas of the filter in Boyden chamber assays. The cell count is proportional to the number of migrated cells. *: p<0.05

In general the therapeutic treatments can be described as following:

### a) Marker for Fibrosis/Parameter for Liver Damage

For the therapy as well as for the prediction of the course of the liver disease and therefore also for the screening- and preventive medical examinations it is important to understand the extent of the liver disease. Important parameter of the hepatic tissue damage is the extent of the inflammation and the extent of the fibrosis. Gold standard and currently the only existing, reliable parameter are the histological examination of for example via biopsy sampled liver tissue. It is desirable, and for the patient considerably less strain full, to be able to analyze relevant serum parameter as reliable indicator for the extend of the hepatic inflammation and fibrosis. Also for the examination during the course of the disease, for example to monitor therapeutic applications, it would be vitally important to have such parameters, since it is not feasible to take several biopsies.

Parameter for the fibrosis with sufficient sensitivity and specificity and applicable in the clinic are not available currently.

Serum transaminases only insufficient or in many cases not at all indicate the extend of the hepatic inflammatory status e.g. for viral chronical liver diseases.

The fibrosis reaction as a correlation to a “scarring” after tissue damage or irritation is in general relatively uniform in most tissues, due to reactions to different noxes. For example, in kidneys, lung, intestine or skin one can observe a fibrosis after chronic inflammation. Also for these diseases and organ systems one can apply similar parameters as for the liver disease: 1) Knowledge about the extent of the fibrosis is important for treatment and prevention strategies and 2) serological parameters would be helpful, but do not exist in the appropriate form.

### b) Tumor Marker

One the worst complication of advanced liver disease is the development of the hepatocellular carcinoma (HCC), which often ends lethal (4^{th} most frequent cause of death for cancer patients). Also, extra-hepatic tumors metastasize frequently into the liver. The screening of such tumors or metastasis is currently done via imaging which is not sensitive enough. The exact diagnosis can only be done after biopsy and histopathological analysis. It would be advantageous to have reliable serum parameters for the screening and diagnosis. Currently, there are no markers for extra-hepatic tumors. In the case of HCC, the only marker is alpha fetoproteine (AFP), which is not reliable due to insufficient sensitivity and specificity (Lun-Xiu Qin, Zhao-You Tang, World J Gastroenterol 2002; 8(3):385-392; Matsumura M et al. J Hepatol 1999; 31:332-339). With this invention and the availability of MIA-2 a new tumor marker is available.

### c) Anti-Fibrotic Therapy

Currently, the only effective anti-fibrotic therapy for chronic liver diseases is the interception of the pathophysiological causes of the disease. But there are no certain therapies which would stop the progression of the hepatic fibrosis in the case of persisting irritation or which would reverse an already apparent fibrosis or cirrhosis of the liver.

As described under a) there is an analogy for other organ systems besides the liver. Effective anti-fibrotic therapeutic approaches are also not available for other organ systems. It is possible that an anti-fibrotic therapeutic approach for the liver can be applied for other organ systems.

Currently there are for the described application areas
a) Marker for fibrosis
b) Marker for hepatic damage/synthesis performance
c) Marker for hepatic tumors and hepatic metastases of extra-hepatic tumors
d) anti-fibrotic therapy
   no sufficient solutions, even those would be urgently needed in the clinic. MIA-2 offers a number of novel approaches for these questions.

Animal studies showed promising results in individual cases and some therapeutic drugs and diagnostic methods are tested in clinical studies. As described above, there are currently no reliable therapies or diagnostic markers available.

### EXAMPLES

### Example 1

### Cloning of MIA-2

### Example 1a

### Cloning of MIA-2 cDNA, Encoding the MIA-2 Protein

For the amplification of the MIA-2 cDNA a RT-PCR with specific primers was performed (SEQ ID NO 3 and SEQ ID NO 4 or SEQ ID NO 9 for the human sequence, and SEQ ID NO 3 and SEQ ID NO 7 for the murine sequence). The RNA was isolated from human or murine liver tissue, transcribed into cDNA using the reverse transcriptase method. This cDNA was applied in a PCR reaction using the appropriate MIA-2 oligonucleotide primer as described above. The PCR product was cloned via blunt-end-ligation into the vector pPCR-Script (Stratagene, catalog Nr. 211188).

### Example 1b

### Cloning of the MIA-2 Promoter

Using specific PCR Primer the MIA-2 promoter was amplified with genomic DNA as template. The amplified fragment was cloned into the Bgl II and Hind III restriction site of the pGL3-basic vector (Promega). For the PCR amplification the following primers were used: SEQ ID NO 10 to SEQ ID NO 17 as “forward” primer and SEQ ID NO 18 as “reverse primer”.

### Example 2

### Recombinant Expression of Human MIA-2 In Vitro

For the in vitro translation MIA-2 cDNA or mutants thereof, which may be more appropriate for specific applications (e.g. more stable, higher affinities to the substrate etc.) was cloned into a eukaryotic expression plasmid system. The vector has besides the motifs necessary for the amplification and stability in E. coli, a T7-promoter and a T7-termination-sequence, as well as appropriate restrictions sites for cloning of the MIA-2 cDNA (e.g. pIVEX2.3-MCS, Roche). In the case of the pIVEX2.3-MCS vector MIA-2 was amplified using the primer according to SEQ ID NO 19 and SEQ ID NO 9 and cloned into the NdeI and Bam HI restriction site of the vector. With commercially available in-vitro-translations systems (e.g. RTS-System, Roche; ECL cell in vitro translation system, Amersham Pharmacia Biotech; PROTEINscript-PRO, Ambion) recombinant MIA-2 proteins was produced. The detection of the specific protein can be done by Western Blot or ELISA using specific antibodies against MLA-2.

### Example 3

### Recombinant Expression of Humane MIA-2 in Eukaryotic Cells

### Example 3a

### Recombinant Expression of MIA-2 in Mammalian Cells

For the expression of MIA-2 in mammalian cells the cDNA of MIA-2, preferable human MIA-2 (SEQ ID NO 1 or SEQ ID 20), is cloned into an appropriate expression vector. This expression vector has an efficient promoter-enhancer system to assure adequate protein production for MIA-2. Such promoters and enhancers are frequently isolated from viruses, for example from SV40, hCMV, polyoma or retroviruses. One can use also other promoters including inducible promoters, like the metallothioneine promoter. The expression vector includes splice acceptor and donor sequences for the RNA processing and a polyA tail for RNA stability. Vectors which are appropriate, are for example pCDNA3 (Invitrogen, San Diego, USA), pCMX-pL1 (Umesono et al., Cell 65 (1991) 1255-1266), or pSG5 (Stratagene, LaJolla, USA). The MIA-2 cDNA can be cloned into a unique restrictions site, for example EcoRI in the case of pCDNA3. The DNA of the expression plasmid containing the MIA-2 cDNA sequence is isolated from Escherichia coli. The mammalian cells are transfected and selected for integration, with appropriate, optimal conditions regarding the expression system and the cell line (see Methods of Enzymology 186 (Gene Expression Technology), ed. David V. Goeddel, Academic Press 1991, Section V). For example the following cell lines can be used to produce recombinant MIA-2 protein: CHO, COS, 3T3, 293 or MelIm cells. MIA-2 protein was detected in the supernatant of the transfected cells and can be used as conditioned media for cell assays of further purified.

### Example 3b

### Recombinant Expression of MIA-2 in Insect Cells

For the expression of MIA-2 in insect cells the cDNA of MIA-2, preferable human MIA-2 (SEQ ID NO 1 or SEQ ID 20), is cloned into an appropriate expression vector, which is derived from AcMNPV (Autographa californica multicapsid nucleopolyhedrosis virus) or BmNPV (Bombyx mori nucleopolyhedrovirus). The MIA-2 cDNA is cloned such that a strong promoter, active in insect cells, regulates the expression. Such a promoter is polH (polyhedrin) or p10 (D. R. O'Reilly, L. K. Miller and V. A. Luckow, Baculovirus expression Vectors—A Laboratory Manual (1992), W. H. Freeman & Co., New York). First the MIA-2 cDNA fragment is cloned into a transfer vector, e.g. pVL1393 (D. R. O'Reilly, L. K. Miller and V. A. Luckow, Baculovirus expression Vectors—A Laboratory Manual (1992), W. H. Freeman & Co., New York). This transfer vector is commercially available and can be amplified in E. coli according to standard methods (Sambrook et al., 1989, Molecular Cloning, A Laboratory Manual, Cold Spring Harbor Press, A Laboratory Manual, Cold Spring Harbor Press, and all successive editions). The transfer of the MIA-2 cDNA from the transfer plasmid to the baculovirus vector occurs via homologous recombination according to routine methods (D. R. O'Reilly, L. K. Miller and V. A. Luckow, Baculovirus expression Vectors—A Laboratory Manual (1992), W. H. Freeman & Co., New York). 0.5 μg BD BaculoGold™ DNA (linearized, modified AcNPV baculovirus DNA with a lethal deletion and lacZ expression, from BD Pharmingen, catalog number 21484P) and 2 μg pVL1393-MIA-2 are mixed, incubated at room temperature for 5 minutes, then mixed with a 1 ml solution of 125 mM Hepes pH 7.1, 125 mM CaCl₂ and 140 mM NaCl. This mixtures is applied to 2×10⁶ SF9 insect cells (Invitrogen, Cat. No. B825-01 or BD Pharmingen Cat. No. 551407) in cell culture dish with a diameter of 60 cm which is covered with 1 ml Grace's Medium plus 10% FBS (fetal bovine serum). After 4 hours at 4° C. the medium is removed and the cells are cultured in fresh medium at 27° C. for 4 days. The obtained recombinant baculovirus are purified twice using the plaque formation assay (D. R. O'Reilly, L. K. Miller and V. A. Luckow, Baculovirus expression Vectors—A Laboratory Manual (1992), W. H. Freeman & Co., New York). Baculovirus containing MIA-2 can be detected by the lack of lacZ expression. With MIA-2 expression recombinant baculovirus SF9 cells are infected after the standard methods (MOI=20 pfu/cell), see also D. R. O'Reilly, L. K. Miller and V. A. Luckow, Baculovirus expression Vectors—A Laboratory Manual (1992), W. H. Freeman & Co., New York.

The cells are incubated for at least 36 hours at 27° C. in serum-free media (e.g. BD BaculoGold Max-XP Insect Cell Medium, BD Pharmingen, Catalog number 551411). Then the cell supernatant is collected and the virus is isolated by ultracentrifugation (Beckmann Ti 60 rotor, 30,000 rpm). The supernatant is filtered through Microcon 100 filter (Amicon, exclusion size of 100 kD). The resulting solution contains the MIA-2 protein which can be either used directly in vitro assays or can be further purified.

### Example 4

### Recombinant Expression of Human Fusion-Free MIA-2 in Escherichia coli

MIA-2 cDNA, preferably human MIA-2 (SEQ ID NO 1 or SEQ ID NO 20) is cloned into an appropriate expression system, for example the T7-expression system from Novagen (Studier and Moffat, J. Mol. Bio. 189 (1986), 113-130) or other systems like pQE40, pGST etc. (e.g. Firma Qiagen, Cat. No. 33403). The MIA-2-cDNA was adapted such that it could be efficiently expressed in E. coli. Depending on the vector MIA-2 can be expressed as a fusion protein with a tag or without.

The expression plasmid was transformed into an appropriate E. coli host, for example BL21 (DE3) E. coli strains which have a sufficient lac repressor expression are inducible and are better suitable. Such a strain is BL21. For the culture of the recombinant bacteria strain a suitable medium, e.g. LB medium in a suitable volume (1.5 l plus 100 μg/ml ampicillin) is used.

### LB-Medium (1 l)

10 g trypton
5 g yeast extract

### 10 g NaCl

The bacterial culture is incubated at 160-200 rpm and 37° C. At an OD₆₀₀ of 0.6 the culture is induced with IPTG and cultured for another 4-5 hours at 37° C. until an OD₆₀₀ of 3 to 3.5 is reached. The cells are harvested by centrifugation at 10,000 g.

The bacterial pellet is resuspended in 2 ml lysis buffer (0.1 M NaPO4, 300 mM NaCl, pH 7.5) and then three times shock frozen and treated with ultrasound for 10 min. The insoluble parts are removed by centrifugation.

The recombinant protein can be purified using chromatographic processes. In the case of a fusion protein, properties of the tag can be used to initially purify MIA-2. After purification the fusion can be cleaved with a suitable protease. MIA-2 protein is analyzed on a 20% SDS-PAGE gel. The protein is stable at −20° C. at least for a month.

### Example 5

### Recombinant Expression of Humane MIA-2 in Escherichia coli as Fusion Protein

The MIA-2 cDNA is cloned into an E. coli expression vector, as described above, see example 1 and example 4. In this case the MIA-2 is flanked by a fusion protein like DHFR, His-Tag or GFP. To cleave MIA-2 proteolytic from the fusion protein a DNA fragment with a recognition site for e.g. the IgA Protease (Ser Arg Pro Pro/Ser) is inserted between fusion protein and MIA-2. The protein expression is done analog to example 4. MIA-2 can be purified using the characteristics of the fusion protein or using the properties of MIA-2 with standard methods. The columns with the bound protein is washed 3 times with lysis buffer and 3 times with wash (z. B. 0.1M Na₂PO₄, 300 mM NaCl, 20% glycerin, pH 6.1). After centrifugation of the column the supernatant is collected and recombinant MIA-2 proteins is analyzed on a 20% SDS-PAGE Gel. The protein can be used for further assays and is stable at −20° C. for at least one month. Die SDS-polyacrylamid-gelelektrophorese-analysis showed that the protein is pure.

### Example 6

### Detection of MIA-2 in Different Tissues and Cell Lines

The expression of MIA-2, preferably of MIA-2 mRNA, can be determined in cells using the commonly used methods of nucleic acid hybridization, e.g. Northern blot analysis, in situ hybridization, dot or slot blot hybridization and derived methods (Sambrook et al., Molecular Cloning—A Laboratory Approach (1989), Cold Spring Harbor Laboratory Press; Nucleic Acid Hybridisation—A practical approach (1985), eds. B. D. Hames and S. J. Higgins, IRL Press; In situ Hybridisation—A practical approach (1993), ed. D. Wilkinson, IRL Press). Also one can determine the MIA-2 expression with specific primers and the RT-PCR (reverse transcriptase polymerase chain reaction) method (PCR Protocols—A guide to Methods and Applications (1990), eds. M. A. Innis, D. H. Gelfand, J J. Sninsky, T. J. White, Academic Press Inc; PCR—A practical approach (1991), eds. M. J. McPherson, P. Quire, G. R. Taylor (1991), IRL Press).

For the in situ hybridization for MIA-2 on tissue sections, a P33-labeled riboprobe containing the 390 N-terminal nucleotides (SEQ ID NO. 23) was produced using standard techniques. After stringent hybridization and stringent wash conditions, the sections were exposed to film for up to 6 days. In FIG. 5 the RNA in situ hybridization shows a specific expression of MIA-2 in the liver of sections of mouse embryos (stage day 12.5 and 14.5). MIA-2 RNA is specifically expressed in the liver.

### Detection of MIA-2 RNA via RT-PCR

FIG. 7 shows MIA-2 RNA expression in several normal human and mouse tissues. For the analysis total RNA was isolated from C57BL/6 mice. The RNA was isolated according to the method of Chomczynski and Sacchi, Anal. Biochem. 162 (1987) 156-159 or using commercially available kits, like RNeasy kit (Qiagen, Hilden, Germany, Cat. no. 75142). About 0.4 cm³ tissue was homogenized in the lysis buffer using shock-freezing and ultrasound. The RNA was separated with RNeasy columns. 1/10 of the obtained RNA was applied in the RT-PCR analysis. The human RNA samples were purchased from Clontech (Heidelberg, Germany) and Ambion (Austin, USA). The RNA was transcribed into cDNA using random dN6 primer and reverse transcriptase. The synthesis of the first strand was done in a volume of 20 μl containing: 2 μg total-RNA, 250 ng dN6 primer (Pharmacia, Freiburg, Germany), 4 μl 5× first strand buffer (Invitrogen Corporation, San Diego, USA), 2 μl 10 mM DTT, 1 μl 10 mM dNTPs and 1 μl Superscript Plus (Invitrogen Corporation, San Diego, USA). The RT-PCR was done semi-quantitatively, and primers for β-actin (SEQ ID NO: 24 and 25) were used as a standard. As standard, also other house-keeping genes can be used, such as hypoxanthine-phosphoribosyl-transferase (HPRT, transferrin receptor, 18S RNA, porphobilinogen deaminase (PBGD), β2-microglobulin, 5-aminolevulinat synthase (ALAS) or glucose-6-phosphate dehydrogenase (GAPDH). Alternatively to the classic or semi-quantitative RT-PCR, the quantitative PCR can be performed using a Lightcyclers (Roche Diagnostics, Mannheim, Germany) or ABI PRISM® 7700 Sequence Detection System (Applied Biosystems, Foster City, Calif., USA).

The classic or semi-quantitative RT-PCR can be performed in any standard PCR thermocycler, like PTC-200 (Biozym, Hess. Oldendorf, Germany) or 96-Well GeneAmp® PCR System 9700 (Applied Biosystems, Foster City, Calif., USA) or any other thermocycler. In a typical RT-PCR reaction 3 μl cDNA were used. The PCR run 32 cycles, and each cycle consists of a denaturing phase (30 sec. at 94° C.), annealing phase (45 sec. at 58-62° C.) and synthesis phase (1 min. at 72° C.). At the end the reaction was incubated at 72° C. for 5 min. The resulting PCR products were electrophoretically separated on a 1.8% agarose gel, stained with ethidium bromide and photographically documented. For MIA-2 specific RT-PCR the following primer were used: for human SEQ ID NO. 3 (MIA-2 forward primer 5′-ATGGCAAAATTTGGCGTTC) and SEQ ID NO. 26 (MIA-2 reverse primer 5′-CCTGCCCACAAATCTTCC) and for mouse SEQ ID NO. 3 (MIA-2 forward primer 5′-ATGGCAAAATTTGGCGTTC) and SEQ ID NO. 7 (MIA-2 reverse primer 5′-CCTGCCCACAAATCTTCT). MIA-2 RNA expression displays the same expression pattern in human and mouse with the most prominent expression in the liver.

Several cell lines were analyzed regarding MIA-2 expression (FIG. 8). MIA-2 RNA is strongly expressed in hepatocytes.

### Example 7

### Functional Studies with MIA-2 on Ito-Cells

To analyze the proliferation of cell lines, the growth was analyzed over 5 days using cell counting. On day −2 the cells were plated at a density of 2×10⁴ cells/well into a 24-well plate. On day 0 the cells were treated with MIA-2 (100 ng/ml-5 μg/ml) or vehicle. Subsequently the cells were counted in duplicates. The proliferation was assessed with a commercially available assay, the XTT assay from Roche (cat. no. 1-465-015). This assay showed that MIA-2 inhibits the proliferation of Ito cells.

### Example 8

### Analysis of MIA-2 Expression in Liver Biopsies

Using the in example 6 descript technology, MIA-2 RNA expression can be analyzed in tissue, tissue fluid, blood and serum of patients. Liver biopsies from patients with different diagnosis were analyzed by RT-PCR. MIA-2 RNA expression was significantly lower in Hepatitis patients with mild fibrosis compared to patients with advanced fibrosis (see FIG. 9)

### Example 9

### rMIA-2 Variants SPR30-03 and SPR30-04 Play a Role in the Inhibition of the Central Mechanism of Liver Fibrosis

Hepatic stellate cells play a key role in the development of liver fibrosis. In response to hepatic injury, hepatic stellate cells (HSC) can transform from a physiologically quiescent cell type (which is mainly characterized by high content of vitamin A storing lipid droplets) into an activated myofibroblast like cell type (which is characterized by loss of vitamin A storage and de novo expression of several pathophysiologically relevant genes). This activation process of hepatic stellate cells is the hallmark of liver fibrosis since activated hepatic stellate cells are the central mediators of hepatic fibrosis in chronic liver disease. These activated cells are the cellular source of the synthesis and excessive deposition of extracellular matrix as it occurs in all chronic liver diseases leading to liver fibrosis. Upon transformation into myofibroblast like cells, increased synthesis of collagen and alpha-smooth muscle actin is observed. Collagen type I is one of the pathophysiologically most relevant extracellular matrix proteins. Further, the activated myofibroblast-like cells express alpha-actin which is a specific marker for the activation of these cells and herewith an essential step in the initiation of liver fibrosis (Bataller & Brenner J. Clin. Invest. 2005, 115, 209-218; Friedman Nat. Clin. Pract. Gastroenterol. Hepatol. 2004, 1, 98-105).

The activation process of hepatic stellate cells can be simulated in vitro by culturing the freshly isolated hepatic stellate cells on plastic cell culture dishes. Within this well characterized and established in vitro model, hepatic stellate cells undergo the same activation process as observed in vivo in response to liver injury. The mRNA expression levels of Collagen Type I (alpha 1) and alpha-smooth muscle actin (alpha-sma) serve as classical and well established markers for the HSC activation.

To test the effect of rMIA2 proteins on the activation process, HSC were stimulated (at day 2 after isolation and cell culture) for 48 h with 800 ng of SPR30-03 and SPR30-04. Subsequently, RNA was isolated by methods known in the art, transcribed in cDNA and analyzed by quantitative PCR (qPCR) for the expression of collagen I and alpha-sma mRNA (FIG. 10).

Treatment of hepatic stellate cells with both MIA-2 fragments leads to a significantly reduced expression of both marker genes for the transformation of HSC into myofibroblast like cells, collagen and alpha-smooth muscle actin. This is a strong indication that both the 20-119 amino acid fragment of MIA-2 as well as the longer 20-253 amino acid fragment of MIA-2 significantly inhibit the activation of hepatic stellate cells. Since activated hepatic stellate cells are the central mediators of hepatic fibrosis in chronic liver, these MIA-2 fragments significantly inhibit the activation of the central mechanism of liver fibrosis. These fragments can therefore be used in therapy and prevention of liver diseases, such as liver fibrosis.

What is claimed is:

1. Human MIA-2 protein, which is encoded by the nucleic acid of SEQ ID NO. 1 or variants thereof, which variants are each defined as having one or more substitutions, insertions, and/or deletions as compared to the nucleic acid of SEQ ID NO. 1, provided that:
these variants hybridize under moderately stringent conditions to a nucleic acid, which comprises the sequence of SEQ ID NO. 1, and further provided that these variants code for a protein having MIA-2 activity; or
b) these variants have nucleic acid changes which are due to the degeneration of the genetic code, which code for the same or functional equivalent amino acid as the nucleic acid of SEQ ID NO. 1.

2. Murine MIA-2 protein, which is encoded by the nucleic acid of SEQ ID NO. 27 or variants thereof, wherein the variants are each defined as having one or more substitutions, insertions and/or deletions as compared to the sequence of SEQ ID NO. 27, provided that:
a) said variants hybridize under moderately stringent conditions to a nucleic acid which comprises the sequence of SEQ ID NO. 27, and further provided that said variants code for a protein having MIA-2 activity; or
b) these variants having nucleic acid changes, which are due to the degeneration of the genetic code, which code for the same or a functional equivalent amino acid as the nucleic acid of SEQ ID NO. 27.

3. An isolated nucleic acid, which comprises the nucleic acid of SEQ ID NO. 1 or variants thereof, wherein the variants are each defined as having one or more substitutions, insertions, and/or deletions as compared to the nucleic acid of SEQ ID NO. 1, provided that:
a) these variants hybridize under moderately stringent conditions to a nucleic acid, which comprises the sequence of SEQ ID NO. 1, and further provided that these variants code for a protein having MIA-2 activity; or
b) said variants have nucleic acid changes which are due to the degeneration of the genetic code, which code for the same or functional equivalent amino acids as the nucleic acid of SEQ ID NO. 1.

4. An isolated nucleic acid which comprises the nucleic acid of SEQ ID NO. 27 or variants thereof, wherein the variants are each defined as having one or more substitutions, insertions, and/or deletions as compared to the sequence of SEQ ID NO. 27, provided that:
a) said variants hybridize under moderately stringent conditions to a nucleic acid, which comprises in the sequence of SEQ ID NO. 27, and further provided that these variants code for a protein having MIA-2 activity; or
b) these variants have nucleic acid changes, which are due to the degeneration of the genetic code, which code for the same or a functional equivalent amino acid as compared to the nucleic acid of SEQ ID NO. 27.

5. The isolated nucleic acid of claim 3 or 4, which is further operably linked to one or more regulatory sequences.

6. The isolated nucleic acid of claim 5, wherein the regulatory sequence is the human MIA-2 promoter of SEQ ID NO. 2.

7. A MIA-2 promoter having the nucleic acid sequence of SEQ ID NO. 2.

8. A nucleic acid, which is a transcriptional product of one of the nucleic acids of claims 3 or 4.

9. A nucleic acid, which selectively hybridizes to transcriptional products of claim 8 under moderately stringent conditions.

10. The nucleic acid of claim 9, which is antisense DNA or RNA.

11. A DNA- or RNA-probe which hybridizes to one of the nucleic acids of claim 3 or 4.

12. The probe of claim 11, comprising the nucleic acid sequence of SEQ ID NO. 23.

13. A primer for the amplification of the nucleic acid of claim 3 or of a transcriptional product thereof, comprising one of the nucleic acids of SEQ ID NOs. 3, 4, 9, or 26.

14. A primer for the amplification of the nucleic acid of claim 4 or of a transcriptional product thereof, comprising one of the nucleic acid of SEQ ID NOs. 3, 7, 9, or 26.

15. A primer for the amplification of the nucleic acid of claim 7, which comprises one of the nucleic acid sequences of SEQ ID NOs. 10-18.

16. Human MIA-2 protein, comprising the amino acid sequence of SEQ ID NO. 5 or a variant of said amino acid sequence, which variant comprises one or more substitution, insertions, and/or deletions as compared to the sequence of SEQ ID NO. 5, and wherein the biological activity of the variant is substantially equal to the activity of the MIA-2 protein, comprising the unmodified amino acid sequence of SEQ ID NO. 5.

17. Murine MIA-2 protein, comprising the amino acid sequence of SEQ ID NO. 28, or a variant of said amino acid sequence, wherein said variant comprises one or more substitutions, insertions, and/or deletions as compared to the amino acid sequence of SEQ ID NO. 28, and wherein the biological activity of the variant is substantially equal to the activity of the MIA-2 protein, comprising the unmodified amino acid sequence of SEQ ID NO. 28.

18. A vector, which comprises the nucleic acid of any of claims 3 or 4.

19. An expression vector, which comprises the nucleic acid sequence of any of claims 3 or 4 and one or more regulatory sequences.

20. The vector of claim 19 which is a plasmid.

21. A host cell, which has been transformed with the vector of claim 19.

22. The host cell of claim 21, which is a eucaryotic cell.

23. The host cell of claim 21, which is a mammalian cell, plant cell, yeast cell, or an insect cell.

24. The mammalian cell of claim 21, which is a CHO-, COS-, HeLa-, 293T-, HEH-, or BHK-cell.

25. The mammalian host cell of claim 21, which is an adult or embryonic stem cell.

26. The host cell of claim 21, which is a procaryotic cell.

27. The host cell of claim 26, which is E. coli or bacillus subtilis.

28. An antibody or an aptamer, which is directed against the MIA-2 protein of claim 16 or 17.

29. The antibody of claim 28, wherein said antibody is selected from the group consisting of a polyclonal antibody, a monoclonal antibody, a humanized antibody, a chimeric antibody, and a synthetic antibody.

30. The antibody of claim 28, which is linked to a toxic agent, and/or to a detectable agent.

31. A hybridoma, which produces a monoclonal antibody having binding specificity for the MIA-2 proteins of claim 16 or 17.

32. A pharmaceutical composition, comprising a therapeutically effective dose of a nucleic acid of claims 3 or 4 or of the vector of claim 18 in combination with a pharmaceutically acceptable carrier.

33. A pharmaceutical composition, comprising a therapeutically effective dose of a protein of claim 16 or 17 in combination with a pharmaceutically acceptable carrier, and optionally in combination with further agents as for example interferons, inhibitors of the ACE-system, or ligands of the proliferation-activated receptor-g (PPAR-g).

34. A pharmaceutical composition, comprising a therapeutically effective dose of an antibody or aptamer of claim 28 in combination with a pharmaceutically acceptable carrier.

35. A diagnostic composition, comprising an antibody or an aptamer of claim 28.

36. A diagnostic composition, comprising the probe of claim 12.

37. A transgenic mouse, in which the nucleic acid of claim 4 has been inactivated.

38. The transgenic mouse of claim 37, in which the nucleic acid of claim 4 has been conditionally inactivated.

39. A transgenic mammal, in the genome of which a nucleic acid of claim 3 or 4 has been inserted.

40. An ex-vivo method for the diagnosis of a liver damage, or for the determination of the hepatic synthesis performance comprising the following steps:
a) providing a liver tissue sample, or a serum sample from a patient;
b) qualitative and/or quantitative determination of transcriptional products of claim 8 in the sample; wherein
an overexpression of the transcriptional products of claim 8 is indicative for a liver damage and/or an enhanced hepatic synthesis performance.

41. The method of claim 40, wherein the determination in step b) is performed by Northern Blot, in situ hybridization or RT-PCR, or a combination thereof.

42. The method of claim 41, wherein an RT-PCR is performed using the primers of claim 13 or 14.

43. The method of claim 40, wherein the determination in step b) is performed by using a composition of claim 35, or 36.

44. The method of one or more of claim 40, wherein the liver damage is a liver cirrhosis, liver fibrosis, or a liver tumor/metastasis.

45. A method of treating fibrosis, comprising administering an effective anti-fibrotic amount of the pharmaceutical composition of claim 32 or 33 to a patient in need of such treatment.

46. A method of treating liver cirrhosis or liver fibrosis, comprising administering an effective anti-cirrhotic or anti-fibrotic amount of the pharmaceutical composition of claim 32 or 33 to a patient in need of such treatment.

47. A method for treating liver tumors/metastasis, comprising administering an therapeutically effective amount of the pharmaceutical composition of claims 32 or 33 to a patient in need of such treatment.

48. A method for producing an organ culture, which method comprises the following steps:
a) providing hepatocytes in a growth media;
b) contacting the mammalian hepatocytes with a MIA-2 protein of claim 16 or 17;
c) isolating the generated organ culture.

49. The method of claim 48, in which human or porcine hepatocytes are used.

50. An organ culture, which is obtainable by the method of claim 48.

51. A method of blood cleansing of a patient, comprising administering an organ culture of claim 50 in a therapeutically effective amount to a patient suffering from an improper liver function.

52. The isolated nucleic acid of claim 3, wherein the variant is defined as bases 1-354 of SEQ ID NO: 1.

53. The isolated nucleic acid of claim 4, wherein the variant is defined as bases 1-357 of SEQ ID NO: 27.

54. Human MIA-2 protein of claim 16, wherein the variant is defined as amino acids 1-118 of SEQ ID NO: 5.

55. Murine MIA-2 protein of claim 17, wherein the variant is defined as amino acids 1-119 of SEQ ID NO: 28.

56. A MIA-2 protein variant of claim 1, which does not comprise amino acids 1 to 19 of SEQ ID NO:1.

57. A MIA-2 protein variant, which is defined by the amino acid sequence of SEQ ID NO:29 or SEQ ID NO:30, or a protein variant which comprises the amino acid according to SEQ ID NO:29 or SEQ ID NO:30 and up to 10, preferably up to 5 additional amino acids at the N- or C-terminus, or a variant of these amino acid sequences, wherein said variants contain one or more substitutions, insertions and/or deletions when compared to the amino acid sequence of SEQ ID NO:29 or SEQ ID NO:30, and wherein the biological activity of the protein variant is at least substantially equal to the activity of the MIA-2 protein.

58. An isolated nucleic acid which is defined by the nucleic acid of SEQ ID NO. 31 or SEQ ID NO:32 or variants thereof, wherein the variants are each defined as having one or more substitutions, insertions, and/or deletions as compared to the sequence of SEQ ID NO. 31 or SEQ ID NO:32, provided that:
said variants hybridize under moderately stringent conditions to a nucleic acid, which comprises in the sequence of SEQ ID NO. 31 or 32, and further provided that these variants code for a protein having MIA-2 activity; or
b) these variants have nucleic acid changes, which are due to the degeneration of the genetic code, which code for the same or a functional equivalent amino acid as compared to the nucleic acid of SEQ ID NO. 31 or 32.


SEQUENCE LISTING (32 sequences)

SEQ ID NO: 1 - DNA, 1626 bp, Homo sapiens
atggcaaaatttggcgttcacagaatccttcttctggctatttctctgacaaagtgtctggagagtacaaaactgctggcagaccttaaaaaatgtggtgacttggaatgtgaagctttaataaacagagtctcagccatgagagattatagaggacctgactgccgatacctgaacttcactaagggagaagagatatctgtttatgttaaacttgcaggagaaagggaagatttgtgggcaggaaaaggaaaggagtttggatattttcccagagatgcagtccagattgaagaggtgttcatatctgaggaaattcagatgtcaacgaaagaatctgactttctttgtcttcttggagtaagttacacatttgacaatgaagatagtgaattaaacggtgattatggtgaaaatatatatccttatgaagaagataaagatgaaaaatctagtatatatgaaagtgattttcagatagaacctggattttatgcaacttatgaaagtactttgtttgaagaccaagttccagcattagaggctcctgaagatatcggaagtaccagtgaatcaaaagactgggaagaagtagttgttgaaagtatggaacaggatcgtattccagaagtgcatgtcccaccatcttcagctgtgtctggagtcaaagaatggtttggattgggaggagaacaagctgaagagaaggcttttgaatcagttattgaacctgtacaagaaagctcatttcggagtagaaaaatagcagtggaagatgagaatgacctagaggaattaaataatggtgagcctcaaacagaacatcagcaagaatctgaatcagaaattgattcagtgccaaagacacagtctgaactagcatctgagtcagagcacattcccaaacctcaatccactggttggtttggtggaggatttacaagttatttaggttttggagatgaggatacagggcttgaattaatagctgaagaaagcaatccaccactacaagattttcccaattccatatcatctgataaagaagccacagttccatgtacagaaatattaacagaaaaaaaagacacaatcactaatgatagcttgagtctcaagccaagttggtttgattttggttttgctatactaggctttgcatatgccaaggaagataaaattatgttagatgacaggaaaaatgaagaagatggtggggcagatgaacatgaacatcctctaacaagtgaattagaccctgaaaaagaacaagaaatagaaacgataaaaattatagaaacagaagatcaaatagacaagaaaccagtctcagaaaaaacagacgaatctgatactataccatatttgaaaaagttcttgtataattttgacaacccttggaacttccagaacattccaaaggaaacagaattgccatttcccaaacagatactggatcaaaataatgtaattgaaaatgaagaaactggagaattttccattgataattatcccacagataatacaaaagttatgatattcaaaagttcatacagtctgtcaggttggtatgaaaatatttacattagaatttattttatttttaaacataatttatttttataa

SEQ ID NO: 2 - DNA, 3090 bp, Homo sapiens
tagcacgagtgtgggagcatttctaataggagcaaatctctccagaccggcatcttaggcaaaaatttttcttgaccgaaatctccctcaggccaatgaataccaaatttagattttgctgggcccttgctgttcttaacttagaacatgattaagtcaattacttctgaatttatccataaattctttatattgtctttaatatttaatacaaaaacaaagagagttaaactacattaattactaaatgttcttgatttaatactggatttggtaatataaaatatgcaaataaccctgagctaaattttgacgctgaagagctgatccttctttaaatgtccttaacaacacagcttgtagatttgcaaaggtactcaagaaactaacctacatcttttctaaaaaaatagagacagggtctcactctgtcacgcagggtggcatgcagtggtgtgacaatagctcactcagcctcaaactcctggggtcaagcaatcctcctgcctcagtctcccaaagtgttgggattacagttgtgaggcacctggcctaacctacacctttttgtgtatgtatgcatacaactgtgcagtgctctgttgcaagccaaattatcctgaaatgcatgctctatcagattcttttgaaggatatcaaaatcttagctctgagagagcgggcttcctactgatcattgttttttgaaaacaaaccaacaaacaaacttcctttacttaggatcttgaaagcaaaagtccttccttcactttagcttgctcgggggctgggtatttgtcctatttgtgtcaggcactagaggggagtgtgacatcatacaagtgctgggttctgacctaagaatcagaagaccaactgtgtgctcactgatatgcccaaatacagtgaaaaagtacagcaaaggagggcttagacaaatattttcccaagaatgaaataggacaaccatggtttctgaaaatcgactgagacctacagttttacccaaaaatttccaatgtgggattttgatcctttttgagtgacggtaaccttcaaatgactggattttcatagctttattttagtatttatttatttattttttcattattgttatttttttgagatggactctctctctgttgcccaggctggagtgcagtggcgcaatctcagctcactgtaacctttgcctcctgggctcaagcgattttcctacctcagcctcccaagtagctgggattacaagtgcctgccacaacacctggctgatttttgtatttttagtagagatggggttttaccatattggccaggctggtcttgaactcctgacctcaggtgatcctcctgccttggctttccaaagtgctgggattacaggtatgagccaccgggcccggctgatttatttttttatttttattttttaattttatttatttatttttttgagacggggtctcgctctgtcgcccaggctagagtgcagtggcaagatcttggctcactgcaagctccgcctcccaggttcaggccattctcctgcctcagccttccgagtagctgggaatacaggcgcccgacaccactcctggctaattttttgtattttttagtagagacggggtttcactgtgttagccaggatggtctcgatctcctgacttcatgatccacccacctcggccttccaaagtgctgggattacagatgtgagccaccgcgcccagctgatttattttaaaataatatacgggccaggtgtggtgactcatgcttgtaatcccagcactttgggaggctgaagcaggaggattacttgagtccaggagttcaagatcagcctggccaacacagtgagacctcatctctactaaaaataaaatattagccagctgtgtcccgcatgcctgtggttcagctactcaggaaggggaggtggaaggattgctcaagcctgggaggcccgcctgggctagagtaagactctgtctcaaaaacaaaacaaaactaaaaaagacatatgctttcttttttaaaaaagaaagccaaacaatataggaaactacaaagaagaaaataaataccaacggaaatcacactgtatacttctaccatgtgggggggcacttattcagatatctctctttcattctctgtccacttaaagaaatatgcatacagtttttaaagcatggagtaatactctacctgctgtttctggagtagtattgttgtagacagagtgacctctttcaatggcagatgacttgattcttggtggtaaagcaagtggttggttataagtatggggtctcattgtgcctaggttaacatttcttttccactcatatgtgccattgagtaagtgatttaacccatctctctgtgcctcagtttccttatacacaaaagtggggacgataatattacctacctcagagagttggtgtgaaggttatatcagatcatatatttaagtacttacttaagacagtacctggaacatagtaagtacttactacatttcaactattgttcttaactaaataagttttaacttcatagtaacaataaggggtttttaaatagaaaagtggaatctttttaaaaatacttttcaccatatttcaaagccattcttttaacataaattattactctttttgcacacagctatatacgttatccactgaaattagtatttgcaggtaactacccttttgcggaggcctacttaaaaactatcctgttgaacacaaggtacctttaagtgagacagttttacatgggcttcctttataacacattagtccaactaaaaatccttgttaattattaaacccttaggtattacaggaatacctgacactgcagattgaaaacagacagtgtttgtctctcaagttaaaccaacaagccgatagaaaaaggtagttatcaagagatttttaaaacttcaaccctttttctcttatagttagtgaagagagtagaatatctccagttttggctgacatctctacaacctgaacaattggcttaaacttcacttgggattcccggttgcttgttttagcatgg

SEQ ID NO: 3 - DNA, 19 bp, Homo sapiens
atggcaaaatttggcgttc

SEQ ID NO: 4 - DNA, 26 bp, Homo sapiens
taaaaataaattatgtttaaaaataa

SEQ ID NO: 5 - protein, 541 aa, Homo sapiens
MAKFGVHRILLLAISLTKCLESTKLLADLKKCGDLECEALINRVSAMRDYRGPDCRYLNFTKGEEISVYVKLAGEREDLWAGKGKEFGYFPRDAVQIEEVFISEEIQMSTKESDFLCLLGVSYTFDNEDSELNGDYGENIYPYEEDKDEKSSIYESDFQIEPGFYATYESTLFEDQVPALEAPEDIGSTSESKDWEEVVVESMEQDRIPEVHVPPSSAVSGVKEWFGLGGEQAEEKAFESVIEPVQESSFRSRKIAVEDENDLEELNNGEPQTEHQQESESEIDSVPKTQSELASESEHIPKPQSTGWFGGGFTSYLGFGDEDTGLELIAEESNPPLQDFPNSISSDKEATVPCTEILTEKKDTITNDSLSLKPSWFDFGFAILGFAYAKEDKIMLDDRKNEEDGGADEHEHPLTSELDPEKEQEIETIKIIETEDQIDKKPVSEKTDESDTIPYLKKFLYNFDNPWNFQNIPKETELPFPKQILDQNNVIENEETGEFSIDNYPTDNTKVMIFKSSYSLSGWYENIYIRIYFIFKHNLFL

SEQ ID NO: 6 - DNA, 1554 bp, Mus sp.
atggcggaagtcagtgttcaaagaatccttcttttggttgtttctctggccaagtgtctggagggtacaaagttgctggcacaccttaagaagtgtggtgacttggaatgtgaaactttgatcagccgagtcttagccctgagagattacacaggacccgactgtcggtacctgaacttcactacgggagaagagatatctgtttatgttaaacttggaggagacagagaagatttgtgggcaggaagcaaaggaaaagactttggattttttcccagagatgcagtcgagattgaagaggtgttcatatctgaagaagtcgaaatgccaactaaatctgactttctttgtcttcttggagaaggctacatatttggaagtgaacagagtgaattaaacagtgaagatgatgaagaacatatgtacccatatgaaaaagatgaagaccaaaactataatatatatgagggtgattttcagccagaacctgacttatatgcagctgctgaagggactttgttggaggaccaaattccagcatccgaagctcctgatgatttccgattctccagtgagtggaaggcctgggaaggggctggaagccagggtggaggggagcaggattacactgcagactctgaccaagacttgccatccctcagtaagccagaaaggcaaggatggtttggcctggggacagaagaagctgaagagaaggttttcgaatcagatactgaacctacacaagaattagcactagaagaggagagtgacctggagaaattacacagtggcgaaccccaagtggaacttgagcaagagccaaaatcagagacattagaattcagttcagtgccggacgaagagtatgagctagaatctgagacggagagtatcctcaaaccccaagcttctggctggtttggtgagggccttacaagttatttaggttttggaaatgaggaggcaggacttgagttattgtccaaagaaagcaatccaccattacaagatattcccagctctgttccaccagatgaagaagtcccggctccatgcagagaaatctcaacagacaaggaagatgcagtcattaatgatagctcggttctcagtccaagctggttttactatggatttggtatgctaggctttacaaatgccgacgaagacaacattgtttcagacaaaggagaaaatgaagatggtgaggtagataacctcaaacatcctataggaagtgactttgaccctgaaaaggaacaagaaaggaaaatagtaactgtggaaaccgaagaccaggcaggtacagaaagcgtcttggagaagacagacgagtctggttccatgcagtatctgaagaagttctttgataatccttggggcttccagagtctcccagaggacacagaattaccattttccaaaaagatgctggatcaagatgatatagtagaaaatgacaaaattgaagaactttccactgaaaattctcccacaggtagcatgaaagaccccgtgatgctggcgagcagatacgttctgtcaggttag

SEQ ID NO: 7 - DNA, 18 bp, Mus sp.
cctgcccacaaatcttct

SEQ ID NO: 8 - DNA, 850 bp, Homo sapiens
ttgtagacagagtgacctctttcaatggcagatgacttgattcttggtggtaaagcaagtggttggttataagtatggggtctcattgtgcctaggttaacatttcttttccactcatatgtgccattgagtaagtgatttaacccatctctctgtgcctcagtttccttatacacaaaagtggggacgataatattacctacctcagagagttggtgtgaaggttatatcagatcatatatttaagtacttacttaagacagtacctggaacatagtaagtacttactacatttcaactattgttcttaactaaataagttttaacttcatagtaacaataaggggtttttaaatagaaaagtggaatctttttaaaaatacttttcaccatatttcaaagccattcttttaacataaattattactctttttgcacacagctatatacgttatccactgaaattagtatttgcaggtaactacccttttgcggaggcctacttaaaaactatcctgttgaacacaaggtacctttaagtgagacagttttacatgggcttcctttataacacattagtccaactaaaaatccttgttaattattaaacccttaggtattacaggaatacctgacactgcagattgaaaacagacagtgtttgtctctcaagttaaaccaacaagccgatagaaaaaggtagttatcaagagatttttaaaacttcaaccctttttctcttatagttagtgaagagagtagaatatctccagttttggctgacatctctacaacctgaacaattggcttaaacttcacttgggattcccggttgcttgttttagcatgg

SEQ ID NO: 9 - DNA, 20 bp, Homo sapiens
aattcactatcttcattgtc

SEQ ID NO: 10 - DNA, 30 bp, Homo sapiens
gacagatctgggtctcattgtgcctaggtt

SEQ ID NO: 11 - DNA, 29 bp, Homo sapiens
gacagatctacccatctctctgtgcctca

SEQ ID NO: 12 - DNA, 29 bp, Homo sapiens
gacagatcttcagagagttggtgtgaagg

SEQ ID NO: 13 - DNA, 29 bp, Homo sapiens
gacagatctcagggtctcactctgtcacg

SEQ ID NO: 14 - DNA, 29 bp, Homo sapiens
gacagatctggaatacctgacactgcaga

SEQ ID NO: 15 - DNA, 27 bp, Homo sapiens
gacaagcttacaagcaaccgggaatcc

SEQ ID NO: 16 - DNA, 33 bp, Homo sapiens
gatccatatgctggagagtacaaaactgctggc

SEQ ID NO: 17 - DNA, 32 bp, Homo sapiens
gcatcggatccttacaagacaaagaaagtcag

SEQ ID NO: 18 - DNA, 28 bp, Homo sapiens
gatgaattcatggcaaaatttggcgttc

SEQ ID NO: 19 - DNA, 29 bp, Homo sapiens
gatgaattctcaaagacaaagaaagtcag

SEQ ID NO: 20 - DNA, 357 bp, Homo sapiens
atggcaaaatttggcgttcacagaatccttcttctggctatttctctgacaaagtgtctggagagtacaaaactgctggcagaccttaaaaaatgtggtgacttggaatgtgaagctttaataaacagagtctcagccatgagagattatagaggacctgactgccgatacctgaacttcactaagggagaagagatatctgtttatgttaaacttgcaggagaaagggaagatttgtgggcaggaaaaggaaaggagtttggatattttcccagagatgcagtccagattgaagaggtgttcatatctgaggaaattcagatgtcaacgaaagaatctgactttctttgtctttga

SEQ ID NO: 21 - DNA, 20 bp, Homo sapiens
atggcaaaatttggcgttca

SEQ ID NO: 22 - DNA, 20 bp, Homo sapiens
ttataaaaataaattatgtt

SEQ ID NO: 23 - DNA, 360 bp, Mus sp.
atggcaaaatttggcgttcacagaatccttcttttggttgtttctctggccaagtgtctggagggtacaaagttgctggcacaccttaagaagtgtggtgacttggaatgtgaaactttgatcagccgagtcttagccctgagagattacacaggacccgactgtcggtacctgaacttcactacgggagaagagatatctgtttatgttaaacttggaggagacagagaagatttgtgggcaggaagcaaaggaaaagactttggatattttcccagagatgcagtccagattgaagaggtgttcatatctgaggaaattcagatgtcaacgaaagaatctgactttctttgtctttaa

SEQ ID NO: 24 - DNA, 25 bp, Homo sapiens
tggaatcctgtggcatccatgaaac

SEQ ID NO: 25 - DNA, 25 bp, Homo sapiens
taaaacgcagctcagtaacagtccg

SEQ ID NO: 26 - DNA, 18 bp, Homo sapiens
cctgcccacaaatcttcc

SEQ ID NO: 27 - DNA, 1628 bp, Mus musculus
atggcggaagtcagtgttcaaagaatccttcttttggttgtttctctggccaagtgtctggagggtacaaagttgctggcacaccttaagaagtgtggtgacttggaatgtgaaactttgatcagccgagtcttagccctgagagattacacaggacccgactgtcggtacctgaacttcactacgggagaagagatatctgtttatgttaaacttggaggagacagagaagatttgtgggcaggaagcaaaggaaaagactttggattttttcccagagatgcagtcgagattgaagaggtgttcatatctgaagaagtcgaaatgccaactaaatctgactttctttgtcttcttggagaaggctacatatttggaagtgaacagagtgaattaaacagtgaagatgatgaagaacatatgtacccatatgaaaaagatgaagaccaaaactataatatatatgagggtgattttcagccagaacctgacttatatgcagctgctgaagggactttgttggaggaccaaattccagcatccgaagctcctgatgatttccgattctccagtgagtggaaggcctgggaaggggctggaagccagggtggaggggagcaggattacactgcagactctgaccaagacttgccatccctcagtaagccagaaaggcaaggatggtttggcctggggacagaagaagctgaagagaaggttttcgaatcagatactgaacctacacaagaattagcactagaagaggagagtgacctggagaaattacacagtggcgaaccccaagtggaacttgagcaagagccaaaatcagagacattagaattcagttcagtgccggacgaagagtatgagctagaatctgagacggagagtatcctcaaaccccaagcttctggctggtttggtgagggccttacaagttatttaggttttggaaatgaggaggcaggacttgagttattgtccaaagaaagcaatccaccattacaagatattcccagctctgttccaccagatgaagaagtcccggctccatgcagagaaatctcaacagacaaggaagatgcagtcattaatgatagctcggttctcagtccaagctggttttactatggatttggtatgctaggctttacaaatgccgacgaagacaacattgtttcagacaaaggagaaaatgaagatggtgaggtagataacctcaaacatcctataggaagtgactttgaccctgaaaaggaacaagaaaggaaaatagtaactgtggaaaccgaagaccaggcaggtacagaaagcgtcttggagaagacagacgagtctggttccatgcagtatctgaagaagttctttgataatccttggggcttccagagtctcccagaggacacagaattaccattttccaaaaagatgctggatcaagatgatatagtagaaaatgacaaaattgaagaactttccactgaaaattctcccacaggtagcatgaaagaccccgtgatgctggcgagcagatacgttctgtcaggttagtatgaaagacttattaatgcacttgttaagatcatttatttttctaagtccaaatcagatgaagatttcgtgct

SEQ ID NO: 28 - protein, 517 aa, Mus musculus
MAEVSVQRILLLVVSLAKCLEGTKLLAHLKKCGDLECETLISRVLALRDYTGPDCRYLNFTTGEEISVYVKLGGDREDLWAGSKGKDFGFFPRDAVEIEEVFISEEVEMPTKSDFLCLLGEGYIFGSEQSELNSEDDEEHMYPYEKDEDQNYNIYEGDFQPEPDLYAAAEGTLLEDQIPASEAPDDFRFSSEWKAWEGAGSQGGGEQDYTADSDQDLPSLSKPERQGWFGLGTEEAEEKVFESDTEPTQELALEEESDLEKLHSGEPQVELEQEPKSETLEFSSVPDEEYELESETESILKPQASGWFGEGLTSYLGFGNEEAGLELLSKESNPPLQDIPSSVPPDEEVPAPCREISTDKEDAVINDSSVLSPSWFYYGFGMLGFTNADEDNIVSDKGENEDGEVDNLKHPIGSDFDPEKEQERKIVTVETEDQAGTESVLEKTDESGSMQYLKKFFDNPWGFQSLPEDTELPFSKKMLDQDDIVENDKIEELSTENSPTGSMKDPVMLASRYVLSG

SEQ ID NO: 29 - protein, 101 aa, Homo sapiens
MLESTKLLADLKKCGDLECEALINRVSAMRDYRGPDCRYLNFTKGEEISVYVKLAGEREDLWAGSKGKEFGYFPRDAVQIEEVFISEEIQMSTKESDFLCL

SEQ ID NO: 30 - protein, 235 aa, Homo sapiens
MLESTKLLADLKKCGDLECEALINRVSAMRDYRGPDCRYLNFTKGEEISVYVKLAGEREDLWAGSKGKEFGYFPRDAVQIEEVFISEEIQMSTKESDFLCLLGVSYTFDNEDSELNGDYGENIYPYEEDKDEKSSIYESDFQIEPGFYATYESTLFEDQVPALEAPEDIGSTSESKDWEEVVVESMEQDRIPEVHVPPSSAVSGVKEWFGLGGEQAEEKAFESVIEPVQESSFRS

SEQ ID NO: 31 - DNA, 300 bp, Homo sapiens
ctggagagtacaaaactgctggcagaccttaaaaaatgtggtgacttggaatgtgaagctttaataaacagagtctcagccatgagagattatagaggacctgactgccgatacctgaacttcactaagggagaagagatatctgtttatgttaaacttgcaggagaaagggaagatttgtgggcaggaagtaaaggaaaggagtttggatattttcccagagatgcagtccagattgaagaggtgttcatatctgaggaaattcagatgtcaacgaaagaatctgactttctttgtctt

SEQ ID NO: 32 - DNA, 702 bp, Homo sapiens
ctggagagtacaaaactgctggcagaccttaaaaaatgtggtgacttggaatgtgaagctttaataaacagagtctcagccatgagagattatagaggacctgactgccgatacctgaacttcactaagggagaagagatatctgtttatgttaaacttgcaggagaaagggaagatttgtgggcaggaagtaaaggaaaggagtttggatattttcccagagatgcagtccagattgaagaggtgttcatatctgaggaaattcagatgtcaacgaaagaatctgactttctttgtcttcttggagtaagttacacatttgacaatgaagatagtgaattaaacggtgattatggtgaaaatatatatccttatgaagaagataaagatgaaaaatctagtatatatgaaagtgattttcagatagaacctggattttatgcaacttatgaaagtactttgtttgaagaccaagttccagcattagaggctcctgaagatatcggaagtaccagtgaatcaaaagactgggaagaagtagttgttgaaagtatggaacaggatcgtattccagaagtgcatgtcccaccatcttcagctgtgtctggagtcaaagaatggtttggattgggaggagaacaagctgaagagaaggcttttgaatcagttattgaacctgtacaagaaagctcatttcggagt